(19) United States
(12) Patent Application Publication
(10) Pub. No.: US 2017/0000804 A1
(43) Pub. Date: Jan. 5, 2017

(54) Methods and Compositions for Inhibiting Mitochondrial Trafficking

(71) Applicant: The Wistar Institute of Anatomy and Biology, Philadelphia, PA (US)

(72) Inventors: Dario C. Altieri, Philadelphia, PA (US); Maria Cecilia Caino, Philadelphia, PA (US)

(73) Assignee: The Wistar Institute of Anatomy and Biology

(21) Appl. No.: 15/192,516

(22) Filed: Jun. 24, 2016

Related U.S. Application Data
(60) Provisional application No. 62/185,370, filed on Jun. 26, 2015.

(51) Int. Cl. A61K 31/585; C12Q 1/68; A61K 31/7088; G01N 33/50; A61K 31/395; C12N 15/113

(52) U.S. Cl. CPC A61K 31/585; A61K 31/395; C12N 15/113; A61K 31/7088; G01N 33/5011; C12Q 1/6886; G01N 33/5079; C12Y 207/01137; C12N 2310/14; C12N 2320/31; C12Q 2600/158; C12Q 2600/178; C12Q 2600/136; G01N 2333/91215

(57) ABSTRACT

Methods for reducing, inhibiting or preventing cancer metastasis comprise blocking the movement of mitochondria within a cancer cell. Other methods involve interrupting or preventing oxidative phosphorylation pathways or respiration pathways in the cancer cell. In one embodiment, mitochondrial movement is induced by contact of the cell with PI3K inhibitors or antagonists. Methods for treating cancer involve a regimen of treating a subject with a PI3K inhibitor or antagonist and treating the subject with a composition that blocks the movement of mitochondria within the subject's cells.

### STATEMENT REGARDING FEDERALLY SPONSORED RESEARCH OR DEVELOPMENT

This invention was made with government support under Grant Nos. P01 CA140043, R01 CA078810, R01 CA190027, P30 CA010815 and W81XWH-13-1-0193 awarded by the National Institutes of Health, National Cancer Institute and Department of Defense. The government has certain rights in this invention.

### BACKGROUND OF THE INVENTION

Molecular therapies are hallmarks of “personalized” medicine, but how tumors adapt to these agents is not well understood. Despite the promise of personalized cancer medicine, most molecular therapies produce only modest and short-lived patient gains. Although this likely involves mechanisms of drug resistance, it is also possible that tumors adaptively reprogram their signaling pathways to evade therapy-induced “stress”, and, in the process, acquire new, more aggressive disease traits. The phosphatidylinositol-3 kinase (PI3K) is a universal tumor driver¹ that integrates growth factor signaling with downstream circuitries of cell proliferation, metabolism and survival². Exploited in nearly every human tumor, including through acquisition of activating mutations³, PI3K signaling is an important therapeutic target, and several small molecule antagonists of this pathway have entered clinical testing⁴. However, the patient response to these agents has been inferior to expectations⁵, dampened by drug resistance⁶, and potentially other mechanisms of adaptation by the tumor⁷.

Small molecule inhibitors of phosphatidylinositol-3 kinase (PI3K), a fundamental cancer node and important therapeutic target, currently in the clinic induce global transcriptional reprogramming and signalling in tumors, with activation of growth factor receptors, (re)phosphorylation of Akt and mammalian target of rapamycin (mTOR), and increased tumor cell motility and invasion. This response involves the trafficking and redistribution of energetically active mitochondria to subcellular sites of cell motility and the cortical cytoskeleton. At this location, the mitochondria provide a potent, “regional” energy source to support tumor cell invasion and membrane dynamics, turnover of focal adhesion complexes and random cell motility. This response may paradoxically increase the risk of metastasis during PI3K therapy.

In this context, there is evidence that therapeutic targeting of PI3K promotes tumor adaptation, paradoxically reactivating Akt in treated cells⁸, and reprograming mitochondrial functions in bioenergetics and apoptosis resistance⁹. How these changes affects tumor traits, however, is unclear. Against the backdrop of a ubiquitous “Warburg effect”¹⁰, where tumors switch from cellular respiration to aerobic glycolysis, a role of mitochondria in cancer has not been clearly defined¹¹, and at times proposed as a “tumor suppressor”¹².

There remains a need in the art for new and effective tools to facilitate treatment of metastatic and refractory cancers and tumors.

### SUMMARY OF THE INVENTION

In one aspect, a method of treating a subject with a cancer or a metastatic cancer involves blocking, reducing, inhibiting or preventing movement of mitochondria within the cancer cell. In one embodiment, the method involves interrupting or preventing oxidative phosphorylation pathways or respiration pathways in the cancer cell. In another embodiment, the method involves blocking, reducing, inhibiting or preventing mitochondrial movement that is induced by contact of the cell with PI3K inhibitors or antagonists.

In another aspect, a method of improving therapeutic outcome in a cancer patient receiving PI3K inhibitors or antagonists is provided. The method includes administering to said patient a composition or regimen that blocks, inhibits, reduces or prevents oxidative phosphorylation or respiration of the cancer cells.

In another aspect, a method of screening molecules for use in cancer therapy comprises contacting a mammalian cancer or tumor cell culture demonstrating mitochondrial trafficking with a test molecule; culturing the cell and examining same for movement of mitochondria; and measuring the oxidative phosphorylation of an selected oxidative pathway, e.g., Akt, mTOR or FAK or respiration of the cell. A decrease or inhibition in mitochondrial trafficking, oxidative phosphorylation or respiration within the cell indicates that the test molecule has an anti-cancer or anti-tumor effect.

A method for screening molecules for carcinogenic potential comprises contacting a healthy mammalian cell culture with a test molecule; culturing the cell culture and examining same for movement of mitochondria; and measuring the oxidative phosphorylation or respiration of the cell. The detection of, or increase in, mitochondrial trafficking, or increase in oxidative phosphorylation or respiration of certain pathways within the cell indicates that the test molecule has a carcinogenic, metastatic, or tumorigenic effect.

Still other aspects and advantages of these compositions and methods are described further in the following detailed description of the preferred embodiments thereof.

### BRIEF DESCRIPTION OF THE DRAWINGS

FIGS. 1A-1G demonstrate PI3K therapy-induced tumor transcriptional reprogramming

FIG. 1A is a heatmap of changes in kinome functions in patient-derived GBM organotypic cultures treated with vehicle or PX-866 (10 μM for 48 h). N, number of genes; %, percent of genes changed for any given function.

FIG. 1B shows extracts from GBM organotypic cultures treated with vehicle (Veh) or PX-866 (10 μM for 48 h) that were incubated with a human phospho-RTK array followed by enhanced chemiluminescence detection. The position and identity of phosphorylated proteins are indicated. M, markers.

FIG. 1C shows PC3 cells that were treated with vehicle (Veh) or the indicated PI3K inhibitors for 48 h and analyzed for changes in Akt activation by Western blotting. p, phosphorylated.

FIG. 1D shows PC3 cells that were treated with vehicle (Veh) or the indicated PI3K inhibitors for 48 h and analyzed for changes in mTOR activation by Western blotting. p, phosphorylated.

FIG. 1E shows primary GBM spheroids treated with vehicle (Veh) or 10 μM PX-866 for 48 h that were imaged by phase contrast and fluorescence microscopy for phosphorylated Akt (Ser473). DNA counterstained with DAPI. Nestin was a GBM marker. p, phosphorylated.

FIG. 1F shows primary GBM spheroids treated with vehicle (Veh) or 10 μM PX-866 for 48 h that were imaged by phase contrast and fluorescence microscopy for phosphorylated mTOR (Ser2448). DNA counterstained with DAPI. Nestin was a GBM marker. p, phosphorylated.

FIG. 1G is a heatmap of changes in kinome pathways in GBM organotypic cultures treated with vehicle or PX-866 (10 μM) for 48 h. N=number of changed genes; Z=z-score of estimated function state: positive numbers=overall function is likely increased; negative numbers=decreased.

FIGS. 2A-2G illustrate that PI3K therapy induces adaptive tumor cell motility and invasion.

FIG. 2A shows tumor cells treated with vehicle (Veh) or 10 μM PX-866 for 48 h and analyzed for invasion across Matrigel-coated Transwell inserts (top panel) or in 3D spheroids (bottom panel). Invasive edges are the outer dark uneven spiky circle surrounding a lighter area which itself surrounds the dark core; the core is represented by the inner dark circle. Representative images. Magnification, 10×.

FIG. 2B illustrates PC3 (top panel) or LN229 (bottom panel) cells that were treated with the indicated increasing concentrations of PX-866 and quantified for invasion across Matrigel (top panel) or in 3D spheroids (bottom panel). The distance between the core and edge of 3D spheroids was determined Mean±SEM of replicates from a representative experiment. *, p=0.02; ***, p<0.0001.

FIG. 2C illustrates patient-derived GBM spheroids that were treated with vehicle (Veh) or PX-866 (0-10 μM) for 48 h and analyzed by phase contrast (top panel) or fluorescence microscopy (bottom panel). The vital dye PKH26 was used to counterstain live GBM neurospheres.

FIG. 2D illustrates membrane ruffling quantified in PC3 cells treated with vehicle (Veh) or PI3K inhibitors for 48 h by SACED stroboscopic microscopy. Average values from at least 330 ruffles per treatment are shown for ruffle size (top panel) and time of ruffle persistence (bottom panel). Mean±SEM (n=15). ***, p<0.0001.

FIG. 2E shows representative stroboscopic images from time lapse video microscopy of PC3 cells treated with vehicle (Veh) or PI3K inhibitors. Four SACED regions corresponding to the top (1), right (2), bottom (3), and (4) of each cell are shown. The ruffling activity (broken lines) is restricted to one main region (1) on the vehicle cell, but distributed equally between 3 regions (1-3) on cells treated with PI3K inhibitors.

FIG. 2F shows PC3 cells that were treated with vehicle (Veh) or PI3K inhibitors and membrane dynamics at lagging areas were quantified. Ruffle size (left panel) or time of ruffle persistence (right panel) from at least 205 individual lagging ruffles are shown. Mean±SEM (n=15). **, p=0.0047; ***, p<0.0001.

FIG. 2G illustrates PC3 cells that were treated with vehicle (Veh) (left panel) or PX-866 (right panel) for 48 h, and quantified for directional vs random cell migration by time-lapse video microscopy (8 h). Rose plots show the distribution of cells migrating along each position interval. Arrows, direction of chemotactic gradient.

FIGS. 3A-3H show that mitochondria fuel focal adhesion dynamics.

FIG. 3A shows PC3 cells treated with vehicle (Veh) or PI3K inhibitors for 48 h that were stained with Mitotracker Red, phalloidin A1exa488 and DAPI, and full cell-stacks were used to generate 3D max projection images that were scored for mitochondrial morphology (polarized, perinuclear, infiltrating).

FIG. 3B are representative confocal 3D max projection images of PC3 cells treated with vehicle (Veh) or the indicated PI3K inhibitors and stained as in FIG. 3A. Bottom panel shows models for quantification of mitochondrial trafficking. Mito, mitochondria. White lines, distance from nuclei to cell border. Striated lines, length of mitochondrial infiltration into membrane lamellipodia. Magnification 63×.

FIG. 3C represent PC3 cells treated with vehicle (Veh) or the indicated PI3K inhibitors that were labeled as in FIG. 3A and quantified for mitochondrial infiltration into lamellipodia. At least 18 cells were analyzed at two independent lamellipodia, and data were normalized to total lamellipodia length. Mean±SEM (n=36). ***, p<0.0001.

FIG. 3D show lung adenocarcinoma A549 or glioblastoma LN229 cells that were labeled as in FIG. 3A and scored for mitochondrial infiltration into membrane lamellipodia by fluorescence microscopy. Mean±SEM. **, p=0.0056; ***, p<0.0001.

FIG. 3E show data for PC3 cells that were transfected with control (Ctrl) or MFN1-directed siRNA, labeled as in FIG. 3A and quantified for mitochondrial infiltration in the cortical cytoskeleton in the presence of vehicle (Veh) or PX-866. Mean±SEM. ***, p<0.0001.

FIG. 3F show PC3 cells that were transfected with control or MFN1-directed siRNA and treated with vehicle (Veh) or PX-866 and analyzed for Matrigel invasion after 48 h. Mean±SEM. p<0.0001.

FIG. 3G shows PC3 cells that were treated with vehicle (Veh) or PI3K inhibitors for 48 h and were replated onto fibronectin-coated slides for 5 h and labeled with an antibody to phosphorylated FAK (pY925) Alexa488, Mitotracker Red and DAPI. Representative 1 μm extended focus confocal images with localization of mitochondria near FA complexes are shown. Magnification 63×.

FIG. 3H show data for PC3 cells that were treated as indicated and quantified for decay, formation and stability of FA complexes per cell over 78 min, n=631.

FIGS. 4A-4L illustrate control of tumor cell invasion by spatio-temporal mitochondria bioenergetics.

FIG. 4A show PC3 cells labeled with Mitotracker Red, phalloidin Alexa488 and DAPI, treated with PX-866 and analyzed for mitochondrial infiltration into the peripheral cytoskeleton in the presence of vehicle (Veh) or mitochondrial-targeted ROS scavenger, mitoTEMPO (mT, 50 μM).

FIG. 4B show data for PC3 cells incubated with the indicated agents, alone or in combination (PX-866+mT) and analyzed for tumor cell invasion across Matrigel. Mean±SEM. p (ANOVA) <0.0001.

FIG. 4C are mtDNA-depleted LN229 (ρ0) cells that were stimulated with NIH3T3 conditioned media for 2 h, labeled with Mitotracker Red, DAPI and phalloidin Alexa488 and analyzed by fluorescence microscopy. Representative pseudo colored images are shown. Magnification 60×.

FIG. 4D are mtDNA-depleted LN229 (ρ0) cells that were stimulated with NIH3T3 conditioned media for 2 h, labeled with Mitotracker Red, DAPI and an antibody to FA-associated paxillin and analyzed by fluorescence microscopy. Representative pseudo colored images are shown. Magnification 60×.

FIG. 4E show WT or ρ0 LN229 cells that were analyzed for invasion across Matrigel-coated Transwell inserts. Representative images of invasive cells stained with DAPI are shown. Magnification, 20×.

FIG. 4F show PC3 cells treated with vehicle (Veh) or PI3K inhibitors in combination with mitochondrial-targeted small molecule Hsp90 inhibitor, Gamitrinib (Gam), labeled with anti-pY925-FAK Alexa488 followed by fluorescence microscopy. Representative 1 μm extended focus confocal images are shown. Magnification 63×.

FIG. 4G show PC3 cells treated with vehicle (Veh) or PI3K inhibitors with or without Gamitrinib (Gam, 1 μM), labeled with Mitotracker Red, phalloidin Alexa488 and DAPI, and quantified after 48 h for mitochondrial infiltration into lamellipodia by fluorescence microscopy, n=48. Mean±SEM. ***, p<0.0009.

FIG. 4H show PC3 cells treated with vehicle (Veh) or PX-866 (5 μM) with or without Gamitrinib (Gam), and quantified for invasion across Matrigel. Mean±SEM of replicates (n=2). ***, p<0.0001.

FIG. 4I show PC3 cells were incubated with vehicle (Veh) or PX-866 alone or in combination with the various mitochondrial respiratory chain inhibitors, and analyzed for Matrigel invasion. Ant A, Antimycin A; Roten, Rotenone; Oligo: Oligomycin. Mean±SEM. **, p=0.006.

FIG. 4J show PC3 cells that were transfected with control siRNA (Ctrl) or siRNA to Akt1/2, mTOR or FAK were labeled as in FIG. 4C, treated with PX-866 and quantified for mitochondrial infiltration into lamellipodia, n=44. Mean±SEM. ***p<0.0001. Ctrl, control siRNA.

FIG. 4K show siRNA-transfected PC3 cells labeled as in FIG. 4C and treated with PX-866 (5 μM) and analyzed for mitochondria morphology (polarized, perinuclear, infiltrating) by fluorescence microscopy, n=21.

FIG. 4L show PC3 cells transfected with the indicated siRNAs that were quantified for invasion across Matrigel in the presence of vehicle (Veh) or PX-866. Mean±SEM (n=4). ***, p<0.0001.

FIGS. 5A-5H show molecular therapy-induced tumor reprogramming FIG. 5A shows extracts from vehicle (Veh)- or PX-866-treated (10 μM for 48 h) LN229 cells that were incubated with a human phospho-RTK array and immunoreactive spots were visualized by enhanced chemiluminescence. The position and identity of phosphorylated proteins are indicated. M, markers.

FIGS. 5B and 5C show data for PC3 cells treated with the indicated PI3K inhibitors and the changes in the levels of the indicated phosphorylated proteins that were quantified by densitometry and normalized to total levels.

FIG. 5D shows PC3 cells that were treated with vehicle (Veh) or the indicated PI3K inhibitors for 48 h and analyzed for invasion across Matrigel. Representative images of invaded cells are shown. Magnification, 10×.

FIG. 5E shows data for the experimental conditions of FIG. 5D in the area occupied by invasive cells. Mean±SEM (n=3). ***, p<0.0001.

FIG. 5F shows data, using the experimental conditions of FIG. 5D for the number of cells before seeding was quantified. Mean±SEM (n=3). ***, p<0.0001.

FIG. 5G shows data for patient-derived GBM spheroids treated with vehicle (Veh) or PX-866 (0-10 μM) for 48 h, stained with the vital dye PKH26, and the number of spheres/field was quantified after 48 h. Mean±SEM (n=5 patients). *, p=0.01; **, p=0.0048; ***, p=0.0004.

FIG. 5H shows data for patient-derived GBM spheroids treated with vehicle (Veh) or PX-866 (0-10 μM) for 48 h, stained with the vital dye PKH26, and the sphere diameter quantified after 48 h. Mean±SEM (n=5 patients). *, p=0.01; **, p=0.0048; ***, p=0.0004.

FIG. 6A-6E illustrate that PI3K therapy stimulates membrane ruffle dynamics.

FIG. 6A shows PC3 cells treated with vehicle (Veh) or the indicated PI3K inhibitors for 48 h and analyzed by time lapse video microscopy. Four discrete regions per cell were followed over time to derive stroboscopic images. Mean±SEM (n=4). Left panels are still images at the beginning of the sequence. White lines indicate the discrete areas analyzed for a cell. Right panels are zoomed in micrographs of the ruffles being analyzed (arrow).

FIG. 6B are PC3 cells treated with vehicle (Veh) or the indicated PI3K inhibitors and analyzed by stroboscopic microscopy. Images representing the dynamics of the cell border over 300 sec are shown. The positions of membrane ruffles and cell body are indicated.

FIG. 6C shows PC3 cells (n=15) as in FIG. 6B that were analyzed for membrane ruffle dynamics by stroboscopic analysis of cell dynamics (SACED) with quantification of average ruffle size. Each bar corresponds to an individual cell. Mean±SEM (n=4).

FIG. 6D shows PC3 cells (n=15) as in FIG. 6B that were analyzed for membrane ruffle dynamics by stroboscopic analysis of cell dynamics (SACED) with quantification of time of ruffle persistence. Each bar corresponds to an individual cell. Mean±SEM (n=4).

FIG. 6E shows PC3 cells (n=15) as in FIG. 6B that were analyzed for membrane ruffle dynamics by stroboscopic analysis of cell dynamics (SACED) with quantification of time of ruffle frequency. Each bar corresponds to an individual cell. Mean±SEM (n=4).

FIGS. 7A-7D show adaptation-induced random tumor cell migration.

FIG. 7A show PC3 cells treated with vehicle or the indicated PI3K inhibitors for 48 h, seeded onto 2D chemotaxis chambers and analyzed by time lapse video microscopy. The sequences were started immediately after setting up a gradient of NIH3T3 conditioned media used as a chemoattractant. Videos were analyzed with WimTaxis software and tracking data exported into the Chemotaxis and Migration tool (Ibidi) for representation. Still images from the time lapse with trajectories overlaid at the indicated time intervals are shown. Scale bar, 50 μm.

FIG. 7B shows data for PC3 cells treated with vehicle or PI3K inhibitors analyzed for 2D chemotaxis. The trajectory (top panel) and end points (bottom panel) represented for all analyzed cells are shown, n≧103. A circular region that splits the Euclidean distances in 50:50 is indicated (radius).

FIG. 7C shows data for PC3 cells treated with vehicle (Veh) or PI3K inhibitors in which membrane dynamics at lagging area were quantified. Ruffle size from at least 205 individual lagging ruffles were analyzed. Mean±SEM (n=15). **, p=0.0047; ***, p<0.0001.

FIG. 7D shows data for PC3 cells treated with vehicle (Veh) or PI3K inhibitors in which membrane dynamics at lagging area were quantified. Time of ruffle persistence from at least 205 individual lagging ruffles was analyzed. Mean±SEM (n=15). **, p=0.0047; ***, p<0.0001.

FIGS. 8A-8H illustrate mitochondrial infiltration to the cortical cytoskeleton.

FIG. 8A shows PC3 cells labeled with Mitotracker Red, phalloidin Alexa488 and DAPI and analyzed by fluorescence microscopy. Representative pseudo colored fluorescence microscopy images of different mitochondrial morphology (polarized, perinuclear, infiltrating) are shown. Magnification 60×.

FIG. 8B shows PC3 cells that were treated with vehicle (Veh) or PI3K inhibitors for 48 h, stained with Mitotracker Red, phalloidin Alexa488 and DAPI, and full cell-stacks used to generate 3D max projection images. Magnification 63×. Scale bar, 10 μm.

FIG. 8C shows the indicated tumor cell types that were treated with vehicle or PX-866 for 48 h, stained as in FIG. 8B, and analyzed by fluorescence microscopy.

FIG. 8D shows PC3 cells that were transfected with control siRNA (Ctrl) or mitofusin 1 (MFN1) or mitofusin 2 (MFN2)-directed siRNA, and analyzed by Western blotting.

FIG. 8E shows data from PC3 cells transfected as in FIG. 8D, which were analyzed for cell viability by Trypan blue exclusion. U, units. Mean±SEM.

FIG. 8F shows data from PC3 cells transfected as in FIG. 8D, which were analyzed for cell viability by normalized ATP production. U, units. Mean±SEM.

FIG. 8G shows PC3 cells transfected with control siRNA (Ctrl) or MFN1-directed siRNA, stained as in FIG. 8B and analyzed by fluorescence microscopy for changes in mitochondrial redistribution. Magnification 60×. Insets were zoomed digitally at 20×. Full cell stacks were post processed for noise reduction using a median filter in LAS AF.

FIG. 8H shows masks that were manually created for cortical mitochondrial quantification, around the periphery of the cell based on the F-actin channel and subsequently applied to the mitochondrial channel to measure intensity at the cortical region. The intensity was normalized to total mitochondrial intensity per cell and background-subtracted. ROI, region of interest.

FIGS. 9A-9F show PI3K therapy regulation of focal adhesion dynamics.

FIG. 9A shows PC3 cells treated with PI3K inhibitor GDC0941 for 48 h and replated onto fibronectin-coated slides for 5 h and labeled with an antibody to phosphorylated FAK (pY925) Alexa488, Mitotracker Red or DAPI. Representative 1 μm extended focus confocal images with localization of mitochondria near pFAK+-focal adhesion complexes (FAs) are shown. Magnification 63×. Scale bar, 10 μm.

FIG. 9B shows LN229 cells that were treated with vehicle (Veh) or PX-866 for 48 h and protein extracts analyzed by Western blotting. p, phosphorylated.

FIG. 9C shows LN229 cells expressing Talin-GFP to label FAs were treated with vehicle (Veh) or PX-866 for 48 h and analyzed by time-lapse microscopy. Representative images at initial (0 min) and final time points (78 min) are merged to visualize FA turnover. Arrows indicate representative decayed (D) or new (N) FAs. Magnification 40×.

FIG. 9D shows data for individual FAs in LN229 cells expressing Talin-GFP as in FIG. 9C, manually tracked to determine FA dynamics The number of dynamic FA per cell were classified into disappearing (decayed) and newly formed (new). Mean±SEM (n=10). *, p=0.0353; ***, p<0.0001.

FIG. 9E shows for individual FAs as in FIG. 9D the turnover rates (number of FAs/h) of FA decay and formation. Mean±SEM (n=10). *, p=0.0235; ***, p<0.0001.

FIG. 9F shows for individual FAs as in FIG. 9D, stable FAs classified into sliding (moving) or mature (>50% overlay between initial and final localization). Mean±SEM (n=10). *p=0.0393.

FIGS. 10A-10F illustrate the role of mitochondrial ROS in organelle trafficking and tumor cell invasion.

FIG. 10A shows PC3 cells treated with vehicle or the indicated PI3K inhibitors, stained with MitoTracker Green FM and MitoSOX Red mitochondrial superoxide probes, and analyzed by fluorescence microscopy. H₂O₂ was used as a control stimulus for ROS production and analyzed by fluorescence microscopy. Menadione (100 μM, 1 h) was used as a control stimulus for ROS production.

FIG. 10B shows the quantification of the PC3 cells of FIG. 10A. Mean±SEM. *P=0.01-0.04; **P=0.002-0.007; P (ANOVA)=0.0002.

FIG. 10C shows data for PC3 cells treated with vehicle (Veh), or PX-866 for 48 h, with or without the mitochondrial-targeted ROS scavenger, mitoTEMPO (mT) and analyzed for ROS levels. Mean±SEM. **, p (ANOVA)=0.0015. ns, not significant.

FIG. 10D shows PC3 cells incubated with vehicle (Veh), mitoTEMPO (mT) alone or in combination with the indicated PI3K inhibitors and analyzed for mitochondrial repositioning to the cortical cytoskeleton by fluorescence microscopy.

FIG. 10E shows quantification of the data of the cells of FIG. 10D. Mean±SEM. p (ANOVA)<0.0001.

FIG. 10F shows data for PC3 cells incubated with vehicle (Veh) or PX-866 for 48 h with or without the indicated concentrations (μM) of pan-ROS scavenger, NAC, and analyzed for Matrigel invasion. Mean±SEM. p (ANOVA)<0.0001.

FIGS. 11A-11E shows the requirement of mitochondrial respiration for FA turnover.

FIG. 11A show respiration competent (WT) LN229 cells incubated with Mitotracker Red, phalloidin Alexa488 and DAPI, and analyzed by fluorescence microscopy. Magnification 60×.

FIG. 11B show respiration competent (WT) LN229 cells incubated with Mitotracker Red, phalloidin Alexa488 and labeled for FAs with an antibody to paxillin, and analyzed by fluorescence microscopy. Magnification 60×.

FIG. 11C shows WT LN229 or mtDNA-depleted LN229 (ρ0) cells expressing Talin-GFP to label FAs that were analyzed by time-lapse microscopy. Representative images at initial (0 min) and final (45 min) time points of the analysis are merged to visualize FA turnover. Representative images of the different stage of FAs (Decayed, Sliding, Stable or New) are indicated.

FIG. 11D shows data for WT or mtDNA-depleted LN229 (ρ0) cells expressing Talin-GFP that were analyzed by time-lapse microscopy, and quantitated for decay, formation and stability of FAs per cell over 45 min, n>431.

FIG. 11E shows data for WT or p0 LN229 cells analyzed for invasion across Matrigel-coated Transwell inserts. Mean±SEM (n=2). ***, p<0.0001.

FIGS. 12A-12F illustrate the requirement of oxidative phosphorylation for mitochondrial infiltration to the cortical cytoskeleton.

FIG. 12A shows data for PC3 cells treated with vehicle (Veh) or PI3K inhibitors in combination with mitochondrial-targeted small molecule Hsp90 inhibitor, Gamitrinib (Gam), labeled with Mitotracker Red, phalloidin Alexa488 and DAPI and scored for mitochondrial morphology (polarized, perinuclear, infiltrating) by fluorescence microscopy (n=42).

FIG. 12B shows data for patient-derived GBM spheroids treated with increasing concentrations of PX-866 (0-10 μM) for 48 h, and imaged by phase contrast microscopy in the presence or absence of Gamitrinib (Gam, 5 μM plus PX-866 at 10 μM). The size of neurospheres was quantified, and normalized on initial size and number per each patient. Mean±SEM (n=4 patients). *, p=0.017, ***, p<0.0001.

FIG. 12C shows data for the number of neurospheres for the data of FIG. 12B quantified, and normalized on initial size and number per each patient. Mean±SEM (n=4 patients). *, p=0.017, ***, p<0.0001.

FIG. 12D shows PC3 cells transfected with control non-targeting siRNA (Ctrl) or TRAP-1-directed siRNA and analyzed by Western blotting.

FIG. 12E shows data for the siRNA-transfected PC3 cells as in FIG. 12D that were labeled with Mitotracker Red, phalloidin Alexa488 and DAPI with quantification of mitochondrial morphology (polarized, perinuclear, infiltrating) by fluorescence microscopy, n=32.

FIG. 12F shows PC3 cells that were labeled as in FIG. 12A, treated with the indicated ETC inhibitors in the presence of PX-866 and analyzed by fluorescence microscopy. Ant. A, Antimycin A. Magnification, 60×.

FIGS. 13A-13G illustrate the requirement of Akt/mTOR signaling for mitochondrial redistribution to cortical cytoskeleton.

FIG. 13A shows PC3 cells that were transfected with control non-targeting siRNA (Ctrl), or siRNA targeting Akt1/2 and analyzed by Western blotting.

FIG. 13B shows PC3 cells that were transfected with control non-targeting siRNA (Ctrl), or siRNA targeting mTOR and analyzed by Western blotting.

FIG. 13C shows PC3 cells that were transfected with control non-targeting siRNA (Ctrl), or siRNA targeting FAK and analyzed by Western blotting.

FIG. 13D shows PC3 cells transfected with control siRNA (Ctrl) or siRNA directed to Akt1/2, mTOR or FAK that were treated with vehicle or PX-866, labeled with Mitotracker red, phalloidin Alexa488 and DAPI, and analyzed by fluorescence microscopy. Representative pseudocolored images are shown. Magnification, 60×.

FIG. 13E shows data for siRNA-transfected PC3 cells that were labeled with Mitotracker Red, phalloidin Alexa488 and DAPI, treated with vehicle (Veh) and scored for mitochondrial infiltration into lamellipodia, n=44 by fluorescence microscopy.

FIG. 13F shows data for siRNA-transfected PC3 cells that were labeled with Mitotracker Red, phalloidin Alexa488 and DAPI, treated with vehicle (Veh) and scored for changes in mitochondrial morphology (polarized, perinuclear, infiltrating), n=25 by fluorescence microscopy.

FIG. 13G shows PC3 cells transfected with wild type (WT) ULK1 or ULK1 S757A mutant cDNA and treated with vehicle (Veh) or PI3K inhibitor PX-866 for 48 h and analyzed for invasion across Matrigel. Trans-migrated cells were stained with crystal violet. A representative image is shown. Magnification, 10×.

### DETAILED DESCRIPTION

The inventors discovered that blocking oxidative phosphorylation prevents adaptive mitochondrial trafficking, impairs membrane dynamics and suppresses tumor cell invasion. Therefore, “spatio-temporal” mitochondrial respiration adaptively induced by PI3K therapy fuels tumor cell invasion, and provides an important anti-metastatic target. Targeting mitochondrial reprogramming provides a novel therapeutic strategy to limit disease dissemination in patients. In the examples herein, the inventors have shown that small molecule PI3K inhibitors currently in the clinic induce global reprogramming of transcriptional and signaling pathways in tumor cells, paradoxically resulting in increased tumor cell motility and invasion. Mechanistically, this involves the trafficking of energetically active mitochondria to the cortical cytoskeleton of tumor cells, where they support membrane lamellipodia dynamics, turnover of FA complexes and random cell migration and invasion. Conversely, interference with this spatio-temporal control of mitochondrial bioenergetics abolishes tumor cell invasion.

Although associated with important tumor traits, including “stemness”¹⁹, malignant regrowth²⁰, and drug resistance²¹, a general role of mitochondria in cancer has been difficult to determine“. Whether these organelles play a role in tumor cell invasion, and, therefore, metastatic competency has been equally controversial, with evidence that mitochondrial respiration is important²², not important²³, or must be dysfunctional²⁴, in order to affect cell movements. The inventors demonstrated in the examples below that disabling cellular respiration with depletion of mitochondrial DNA²⁵, or targeting oxidative phosphorylation complex(es)¹⁸, prevented mitochondrial trafficking to the cortical cytoskeleton, abolished membrane dynamics of cell motility, and suppressed cell invasion. Conversely, scavenging of mitochondrial ROS, which are increased in response to PI3K therapy, did not affect organelle dynamics and tumor cell invasion. Together, these data provide evidence that oxidative phosphorylation contributes to cancer metabolism, and provides a “regional” and potent ATP source to fuel highly energy-demanding processes of cell movements and invasion²⁶.

This “spatio-temporal” model of mitochondrial bioenergetics is reminiscent of the accumulation of mitochondria at subcellular sites of energy-intensive processes in neurons²⁷, including synapses, active growth cones, and branches²⁸. The cytoskeletal machinery that transports mitochondria along the microtubule network in neurons²⁹ may also be exploited in cancer. However, comparable mechanisms of organelle dynamics³⁰ support mitochondrial redistribution in lymphocytes³¹, and may contribute to directional migration of tumor cells³². Interference with the mitochondrial fusion machinery, i.e. mitofusins suppressed mitochondrial repositioning to the cortical cytoskeleton and tumor cell invasion mediated by PI3K therapy.

In addition to oxidative phosphorylation, Akt-mTOR signaling was identified here as a key regulator of mitochondrial trafficking and tumor cell invasion. This is consistent with a pivotal role of PI3K in directional cell movements³³, supporting chemotaxis at the leading edge of migration³⁴, and Rac1 activation³⁵. A third signaling requirement of this pathway involved FAK activity¹⁷, which has also been implicated in cytoskeletal dynamics³⁶.

The data described herein suggest that PI3K antagonists and inhibitory agents potently activate global adaptive mechanisms in tumors⁷, unexpectedly centered on mitochondrial reprogramming in cell survival/bioenergetics⁹, and subcellular trafficking. In this context, the increased tumor cell motility and invasion stimulated by PI3K inhibitors creates an “escape” mechanism for tumor cells to elude therapy-induced environmental stress, reminiscent of the heightened metastatic propensity associated with other unfavorable conditions of hypoxia³⁷, acidosis³⁸, and anti-angiogenic therapy^{39,40}. Although this adaptive response to PI3K therapy may paradoxically promote more aggressive tumor traits, and further compromise clinical outcomes, disabling mitochondrial adaptation and trafficking without the cell provide a viable strategy to increase the anticancer efficacy of PI3K antagonists in the clinic. As described herein and exemplified by the data in the examples below, the inventors have demonstrated and shown supportive evidence that the impact of mitochondrial reprogramming induced by PI3K therapy on mechanisms of tumor progression can be counteracted to reduce the metastatic growth of tumors.

Technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The terms “a” or “an” refers to one or more, for example, “an inhibitor” is understood to represent one or more such compounds, molecules, peptides or antibodies. As such, the terms “a” (or “an”), “one or more,” and “at least one” are used interchangeably herein. As used herein, the term “about” means a variability of 10% from the reference given, unless otherwise specified. While various embodiments in the specification are presented using “comprising” language, under other circumstances, a related embodiment is also intended to be interpreted and described using “consisting of” or “consisting essentially of” language.

As used herein, the term “subject” as used herein means a multicellular and/or mammalian animal, including a human, a veterinary or farm animal, a domestic animal or pet, and animals normally used for clinical research. In one embodiment, the subject of these methods and compositions is a human. Still other suitable subjects include, without limitation, murine, rat, canine, feline, porcine, bovine, ovine, and others.

The term “neoplastic disease”, “cancer” or “proliferative disease” as used herein refers to any disease, condition, trait, genotype or phenotype characterized by unregulated or abnormal cell growth, proliferation or replication. The abnormal proliferation of cells may result in a localized lump or tumor, be present in the lymphatic system, or may be systemic. In one embodiment, the neoplastic disease is benign. In another embodiment, the neoplastic disease is pre-malignant, i.e., potentially malignant neoplastic disease. In a further embodiment, the neoplastic disease is malignant, i.e., cancer. In still a further embodiment the neoplastic disease is a refractory cancer. In another embodiment, the cancer is a metastasis of an original cancer.

In various embodiments of the methods and compositions described herein, the cancer can include, without limitation, breast cancer, lung cancer, prostate cancer, colorectal cancer, brain cancer, esophageal cancer, stomach cancer, bladder cancer, pancreatic cancer, cervical cancer, head and neck cancer, ovarian cancer, melanoma, leukemia, myeloma, lymphoma, glioma, and multidrug resistant cancer. In another embodiment, the neoplastic disease is Kaposi's sarcoma, Merkel cell carcinoma, hepatocellular carcinoma (liver cancer), cervical cancer, anal cancer, penile cancer, vulvar cancer, vaginal cancer, neck cancer, head cancer, multicentric Castleman's disease, primary effusion lymphoma, tropical spastic paraparesis, adult T-cell leukemia, Burkitt's lymphoma, Hodgkin's lymphoma, post-transplantation lymphoproliferative disease, nasopharyngeal carcinoma, pleural mesothelioma (cancer of the lining of the lung), osteosarcoma (a bone cancer), ependymoma and choroid plexus tumors of the brain, and non-Hodgkin's lymphoma. In still other embodiments, the cancer may be a systemic cancer, such as leukemia. In one aspect, the cancer is a human glioblastoma. In another aspect, the cancer is a prostate adenocarcinoma. In still another embodiment, the cancer is a lung adenocarcinoma. In one embodiment, the cancer is non-small cell lung adenocarcinoma (NSCLC). In another embodiment, the cancer is squamous cell carcinoma. In another embodiment, the cancer is liver cancer. In another embodiment, the cancer is a breast adenocarcinoma. In still another embodiment, the cancer is melanoma. In another example, the cancer is a multidrug resistant cancer. In one embodiment, the cancer is a drug resistant cancer.

A “cancer cell” is cell that divides and reproduces abnormally with uncontrolled growth. This cell can break away from the site of its origin (e.g., a tumor) and travel to other parts of the body and set up another site (e.g., another tumor), in a process referred to as metastasis. A “tumor” is an abnormal mass of tissue that results from excessive cell division that is uncontrolled and progressive, and is also referred to as a neoplasm. Tumors can be either benign (not cancerous) or malignant.

The term “regulation”, “inhibition” or variations thereof as used herein refers to the ability of a compound of a compound or composition described herein to inhibit, retard, suppress or otherwise effect the expression, phosphorylation or activity of one or more components of a biological pathway that normally leads to mitochondrial trafficking.

As used herein, “disease”, “disorder” and “condition” are used interchangeably, to indicate an abnormal state in a subject.

The term “treating” or “treatment” is meant to encompass administering to a subject a compound described herein for the purposes of amelioration of one or more symptoms of a disease or disorder.

In one embodiment as exemplified by the data in the examples and figures, the methods of inhibiting mitochondrial trafficking within the cell or without the cell are practiced when the subject has an established malignancy, or a refractory cancer, or a metastatic cancer. Similarly such methods are useful when the subject is newly diagnosed and prior to treatment. These methods, in another embodiment, involve administration of one or more compositions that inhibit mitochondrial trafficking, at a selected time during the course of cancer therapy. In one such embodiment, the method further includes administering blockers or inhibitors of mitochondrial trafficking as part of a therapeutic regimen involving the administration of PI3K inhibitors or antagonists.

“Target pathway” or “target pathway gene/protein” as used herein refers to one of the pathway genes or proteins described herein, including mitofusin-1 or mitofusin-2, TRAP-1, mTOR, FAK, AMK, mito-Complex I, mito-Complex III, mito-Complex V, mitochondrial uncoupler CCCP, Akt1, Akt2, or others identified in the examples below.

The term “inhibitor or antagonist” of one of the target pathway genes or proteins described herein includes small chemical/pharmaceutical molecules, peptides, nucleotide sequences, e.g., siRNA or shRNAs, and intracellular antibodies that have the ability to penetrate the cell and bind or interact with the targeted pathway gene or its expression product so as to prevent or inhibit the expression or activity of that target. This inhibition suppresses or retards for a certain time period the oxidative phosphorylation or respiration pathway and inhibits mitochondrial movement.

Small molecule inhibitors or antagonists include those known in the art to antagonize the indicated targets and their salts derived from pharmaceutically or physiologically acceptable acids, bases, alkali metals and alkaline earth metals. Physiologically acceptable acids include those derived from inorganic and organic acids. A number of inorganic acids are known in the art and include, without limitation, hydrochloric, hydrobromic, hydroiodic, sulfuric, nitric, and phosphoric acid. A number of organic acids are also known in the art and include, without limitation, lactic, formic, acetic, fumaric, citric, propionic, oxalic, succinic, glycolic, glucuronic, maleic, furoic, glutamic, benzoic, anthranilic, salicylic, tartaric, malonic, mallic, phenylacetic, mandelic, embonic, methanesulfonic, ethanesulfonic, panthenoic, benzenesulfonic, toluenesulfonic, stearic, sulfanilic, alginic, and galacturonic acids Inhibitor compound salts can be also in the form of esters, carbamates, sulfates, ethers, oximes, carbonates, and other conventional “pro-drug” forms, which, when administered in such form, convert to the active moiety in vivo. In one embodiment, the prodrugs are esters. The inhibitor compounds discussed herein also encompass “metabolites” which are unique products formed by processing the selected inhibitor compound by the cell or subject. In one embodiment, metabolites are formed in vivo.

Also useful in suppressing or down-regulating the expression, activity or phosphorylation of the target of the inhibitors are “antisense” nucleotide sequence or a small nucleic acid molecule having a complementarity to a target nucleic acid sequence, e.g., TRAP1, FAK, MFN, mito-ComplexIll, mTORC1, AMK, or Akt, etc. It can also comprise a nucleic acid sequence having complementarity to a sense region of the small nucleic acid molecule. For example, in one embodiment the composition comprises a nucleic acid construct comprising a sequence that reduces or suppresses the expression of one of the targets or a combination thereof in the target cancer cells. For example, the inhibiting composition can include a nucleic acid construct comprising a short nucleic acid molecule selected from the group consisting of a short hairpin RNA (shRNA), a short interfering RNA (siRNA), a double stranded RNA (dsRNA), a micro RNA, and an interfering DNA (DNAi) molecule, optionally under the control of a suitable regulatory sequence.

The term “intracellular antibody” defines an antibody, particularly a non-naturally occurring antibody, or fragment of an antibody that has the ability to penetrate cell membranes and act intracellularly. See, e.g., Perez-Martinez et al, July 2010 BioEssays, 32(7):589-598 and references cited therein.

“PI3K inhibitors or antagonists” are the PI3K inhibitor compounds described herein and employed in the examples below and other molecules shown to antagonize or inhibit PI3K. In one embodiment, the PI3K inhibitor is PX-866. In another embodiment, the PI3K inhibitor is AZD6482. In another embodiment, the PI3K inhibitor is LY294002. In another embodiment, the PI3K inhibitor is BEZ235. In another embodiment, the PI3K inhibitor is GSK458. In another embodiment, the PI3K inhibitor is GDC0941. In another embodiment, the PI3K inhibitor is ZSTK474. In another embodiment, the PI3K inhibitor is BKM120. In another embodiment, the PI3K inhibitor is GSK2636771. In another embodiment, the PI3K inhibitor is GSK458. In another embodiment, the PI3K inhibitor is GDC-0980. Still other PI3K inhibitors or antagonists are expected to be useful in these methods.

By the term “mito-complex inhibitors or antagonists” is meant the mito-Complex I, mito-Complex III and mito-Complex V inhibitor compounds described herein and employed in the examples below and other molecules shown to antagonize or inhibit the expression or activity or phosphorylation of these targets. In one embodiment, an inhibitor of mito-Complex I is e.g., Rotenone or Amytal. In one embodiment, an inhibitor of mito-Complex III is e.g., AnimycinA. In one embodiment, an inhibitor of mito-Complex V is e.g., Oligomycin. Other examples include atpenin A5, harzianopryidone or venturicidin, listed in Enzo Life Science.com product listing.

By the term “mitofusin-1 or mitofusin-2 inhibitors or antagonists” is meant mitofusin-1 or mitofusin-2 inhibitor compounds described herein and employed in the examples below and other molecules shown to antagonize or inhibit the expression or activity or phosphorylation of these targets.

“TRAP-1 inhibitors or antagonists” refer toTRAP-1 inhibitor compounds described herein and employed in the examples below and other molecules shown to antagonize or inhibit the expression or activity or phosphorylation of these targets. Examples of TRAP-1 inhibitors are NVP-BEP800 and Luminespib, identified in Selleckchem.com, Inhibitor catalog, among others.

“mTOR inhibitors or antagonists” refer to mTOR inhibitor compounds described herein and employed in the examples below and other molecules shown to antagonize or inhibit the expression or activity or phosphorylation of these targets.

“FAK inhibitors or antagonists” refer to FAK inhibitor compounds described herein and employed in the examples below and other molecules shown to antagonize or inhibit the expression or activity or phosphorylation of these targets. Examples of FAK inhibitors includes PF-00562271, TAE226, PF-573228, and Defactinib, identified in Selleckchem.com, Inhibitor catalog, among others.

“AMK inhibitors or antagonists” refer to AMK inhibitor compounds described herein and employed in the examples below and other molecules shown to antagonize or inhibit the expression or activity or phosphorylation of these targets.

“CCCP inhibitors or antagonists” refer to CCCP inhibitor compounds described herein and employed in the examples below and other molecules shown to antagonize or inhibit the expression or activity or phosphorylation of these targets.

“Ack1 or Ack2 inhibitors or antagonists” refer to Ack1 or Ack2 inhibitor compounds described herein and employed in the examples below and other molecules shown to antagonize or inhibit the expression or activity or phosphorylation of these targets. Examples of Ack inhibitors are perifosine, MK2206, GSK2141795, GSK690693, ipatasertib, AZD5363, AT7867, AFURESERTIB, AT3148, among other known inhibitors. See, e.g., Selleckchem.com inhibitor catalog, Akt inhibitor list, among others.

Still other inhibitors useful in the methods described herein may be found in the catalogs of various biochemical and pharmaceutical suppliers.

In one aspect, a method of treating or inhibiting cancer growth or metastasis comprises blocking, reducing, inhibiting or preventing movement of mitochondria within the cancer cell. In one embodiment, the inhibition of mitochondrial movement involves contacting the cell with, or administering to a subject in need of such treatment, a composition or molecule that interrupts or inhibits oxidative phosphorylation pathways or respiration pathways in the cancer cell. In one embodiment, the mitochondrial movement is induced by contact of the cell with PI3K inhibitors or antagonists.

In one embodiment of the method, the subject is administered a composition or molecule that acts to down-regulate, inhibit, suppress or eliminate the expression or activity of mitofusin-2 (MFN-2) in a cancer cell in the presence of a PI3K inhibitor or antagonist. In another embodiment, the subject is administered a composition or molecule that acts to down-regulate, inhibit, suppress or eliminate the expression or activity of TRAP-1 in the presence of a PI3K inhibitor or antagonist. In still another embodiment, the subject is administered a composition or molecule that acts to down-regulate, inhibit, suppress or eliminate the expression or activity of mTOR in the presence of a PI3K inhibitor or antagonist.

In still other embodiments, the methods of treating a cancer by inhibiting the mitochondrial trafficking with the cancer cell involves down-regulating, inhibiting, suppressing or eliminating the expression or activity of FAK in the presence of a PI3K inhibitor or antagonist. In still other embodiments, the methods of treating a cancer by inhibiting the mitochondrial trafficking with the cancer cell involves down-regulating, inhibiting, suppressing or eliminating the expression or activity of AMK in the presence of a PI3K inhibitor or antagonist.

In still other embodiments, the methods of treating a cancer by inhibiting the mitochondrial trafficking with the cancer cell involves down-regulating, inhibiting, suppressing or eliminating the expression or activity of mito-Complex I in the presence of a PI3K inhibitor or antagonist. In still other embodiments, the methods of treating a cancer by inhibiting the mitochondrial trafficking with the cancer cell involves down-regulating, inhibiting, suppressing or eliminating the expression or activity of mito-Complex III in the presence of a PI3K inhibitor or antagonist. In still other embodiments, the methods of treating a cancer by inhibiting the mitochondrial trafficking with the cancer cell involves down-regulating, inhibiting, suppressing or eliminating the expression or activity of mito-Complex V in the presence of a PI3K inhibitor or antagonist.

In still other embodiments, the methods of treating a cancer by inhibiting the mitochondrial trafficking with the cancer cell involves down-regulating, inhibiting, suppressing or eliminating the expression or activity of mitochondrial uncoupler CCCP in the presence of a PI3K inhibitor or antagonist. In still other embodiments, the methods of treating a cancer by inhibiting the mitochondrial trafficking with the cancer cell involves down-regulating, inhibiting, suppressing or eliminating the expression or activity of Akt1 or Akt2 in the presence of a PI3K inhibitor or antagonist. In still other embodiments, the methods of treating a cancer by inhibiting the mitochondrial trafficking with the cancer cell involves down-regulating, inhibiting, suppressing or eliminating the expression or activity of Akt2 in the presence of a PI3K inhibitor or antagonist.

Similarly the methods of this invention may employ such compositions directed to other molecules in the pathways described herein.

According to these methods, the PI3K antagonists and one of the above-noted mitochrondrial trafficking inhibitors, noted above, are administered prior to or during a course of chemotherapy or radiation to reduce the size of an existing primary tumor, to prevent or retard mitochondrial movement in the cells so treated. In another embodiment, the methods involve administering a dose of the PI3K antagonists and one of the above-noted mitochrondrial trafficking inhibitors prior to or during surgery performed to decrease mitrochondrial trafficking during tumor removal. In still another embodiment, the methods comprise administering the PI3K antagonists and one of the above-noted mitochrondrial trafficking inhibitors after surgery. In yet a further embodiment, the methods involve administering the PI3K antagonists and one of the above-noted mitochrondrial trafficking inhibitors prior to or during a second or repeated course of chemotherapy or radiation. In certain embodiments, the second or repeated course is post-surgery. Still further embodiments of the methods described herein include administering a continuous course of a dose of the PI3K antagonist with continuous or intermittent administrations of the compounds that inhibit the mitochondrial trafficking to a subject in need thereof. In other embodiments the methods of treatment involve administering a course of PI3K antagonists followed subsequently by a course of the selected mitochondrial trafficking inhibitor. Such courses of therapy may be repeated. Still a further embodiment of the method includes administering an intermittent course of a dose of the PI3K antagonist with intermittent administrations of the compounds that inhibit the mitochondrial trafficking to a subject in need thereof. Other regimens which balance the effects of the PI3K antagonists with the effects of the inhibitors of mitochondrial trafficking may be selected by the attending physician based upon the condition and responsiveness of the subject to the therapy.

The dosage required for a either or both of the PI3K antagonists and the inhibitors of mitochondrial trafficking depends primarily on factors such as the condition being treated, the age, weight and health of the patient, and may thus vary among patients. The effective dosage of each active component is generally individually determined, although the dosages of each compound can be the same. In one embodiment, the small molecule dosage is about 1 μg to about 1000 mg. In one embodiment, the effective amount is about 0.1 to about 50 mg/kg of body weight including any intervening amount. In another embodiment, the effective amount is about 0.5 to about 40 mg/kg. In a further embodiment, the effective amount is about 0.7 to about 30 mg/kg. In still another embodiment, the effective amount is about 1 to about 20 mg/kg. In yet a further embodiment, the effective amount is about 0.001 mg/kg to 1000 mg/kg body weight. In another embodiment, the effective amount is less than about 5 g/kg, about 500 mg/kg, about 400 mg/kg, about 300 mg/kg, about 200 mg/kg, about 100 mg/kg, about 50 mg/kg, about 25 mg/kg, about 10 mg/kg, about 1 mg/kg, about 0.5 mg/kg, about 0.25 mg/kg, about 0.1 mg/kg, about 100 μg/kg, about 75 μg/kg, about 50 μg/kg, about 25 μg/kg, about 10 μg/kg, or about 1 μg/kg. However, the effective amount of the PI3K inhibitor and the mitochondrial trafficking inhibitor compound, when employed simultaneous or sequentially, or in any other regimen, as well as suboptimal doses lower than ordinarily used for therapy, can be determined by the attending physician and depends on the condition treated, the compound administered, the route of delivery, age, weight, severity of the patient's symptoms and response pattern of the patient.

Toxicity and therapeutic efficacy of the compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are preferred. While compounds that exhibit toxic side effects may be used, care should be taken to design a delivery system that targets such compounds to the site of affected tissue, e.g., bone or cartilage, in order to minimize potential damage to uninfected cells and, thereby, reduce side effects.

The data obtained from cell culture assays (such as those described in the examples below) and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the method of the invention, the therapeutically effective dose can be estimated initially from cell culture assays. A dose may be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

One or more of the PI3K inhibitors and the selected mitochondrial trafficking inhibitors discussed herein may be administered in combination with other pharmaceutical agents, as well as in combination with each other. The term “pharmaceutical” agent as used herein refers to a chemical compound which results in a pharmacological effect in a patient. A “pharmaceutical” agent can include any biological agent, chemical agent, or applied technology which results in a pharmacological effect in the subject.

The therapeutic compositions administered by these methods are administered directly into the environment of the targeted cell undergoing unwanted proliferation, e.g., a cancer cell or targeted cell (tumor) microenvironment of the patient. Conventional and pharmaceutically acceptable routes of administration include, but are not limited to, systemic routes, such as intraperitoneal, intravenous, intranasal, intravenous, intramuscular, intratracheal, subcutaneous, and other parenteral routes of administration or intratumoral or intranodal administration. Routes of administration may be combined, if desired. In some embodiments, the administration is repeated periodically.

These therapeutic compositions, i.e., PI3K inhibitors only, mitochondrial trafficking inhibitors only, or a combination of both inhibitors in a single composition, may be administered to a patient, preferably suspended in a biologically compatible solution or pharmaceutically acceptable delivery vehicle. The various components of the compositions are prepared for administration by being suspended or dissolved in a pharmaceutically or physiologically acceptable carrier such as isotonic saline; isotonic salts solution or other formulations that will be apparent to those skilled in such administration. The appropriate carrier will be evident to those skilled in the art and will depend in large part upon the route of administration. Other aqueous and non-aqueous isotonic sterile injection solutions and aqueous and non-aqueous sterile suspensions known to be pharmaceutically acceptable carriers and well known to those of skill in the art may be employed for this purpose.

In still another aspect, a method of improving therapeutic outcome in a cancer patient receiving PI3K inhibitors or antagonists comprising administering to said patient a composition or regimen, such as those described above that blocks oxidative phosphorylation or respiration of the cell. In one embodiment, oxidative phosphorylation or respiration blocking therapy is administered concurrently with the PI3K therapy. In another embodiment, oxidative phosphorylation or respiration blocking therapy is administered sequentially with the PI3K therapy. In still another embodiment said oxidative phosphorylation or respiration blocking therapy is administered at specific time points within the PI3K therapeutic regimen.

In yet another aspect, a method of screening molecules for use in cancer therapy comprises contacting a mammalian cancer or tumor cell culture demonstrating mitochondrial trafficking with a test molecule, e.g., a small molecule, peptide, nucleotide sequence, intracellular antibody or the like; and culturing the cell. The culture is then examined for movement of mitochondria. Thereafter, the oxidative phosphorylation of an AKT pathway, an mTOR pathway or a FAK pathway or respiration of the cell is measured. In one embodiment, a decrease or inhibition in mitochondrial trafficking, oxidative phosphorylation or respiration within the cell indicates that the test molecule has an anti-cancer or anti-tumor effect. In another embodiment, the mitochondrial trafficking is PI3K antagonist-induced by contacting the cell culture with a PI3K antagonist prior to contact with the test molecule. In this embodiment, a decrease or inhibition in mitochondrial trafficking, oxidative phosphorylation or respiration within the cell indicates that the test molecule has an anti-cancer or anti-tumor effect in concert with the PI3K antagonist.

The decrease of inhibition in the test cell culture can be compared to the level of mitochondrial trafficking or oxidative phosphorylation or cell respiration in untreated normal cell cultures. In another embodiment, the decrease of inhibition in the test cell culture can be compared to the level of mitochondrial trafficking or oxidative phosphorylation or cell respiration in untreated cancer/tumor cell cultures. In another embodiment, the decrease of inhibition in the test cell culture can be compared to the level of mitochondrial trafficking or oxidative phosphorylation or cell respiration in cancer/tumor cell cultures previously treated with a PI3K inhibitor or exposed to, or treated with some other cancer therapy. In another embodiment, the decrease of inhibition in the test cell culture can be compared to the level of mitochondrial trafficking or oxidative phosphorylation or cell respiration in cancer/tumor cell cultures established from refractory cancer cells or cultures established from known metastatic cancer cells, and the like.

In another embodiment, a method for screening molecules for carcinogenic potential comprises contacting a healthy mammalian cell culture with a test molecule, e.g., a small molecule, peptide, nucleotide sequence, intracellular antibody or the like; culturing the cells and examining the cell culture for movement of mitochondria. The oxidative phosphorylation of the pathways described herein and/or the respiration of the cell culture is measured. The detection of, or increase in, mitochondrial trafficking, or increase in oxidative phosphorylation or respiration of the pathways identified herein within the cell indicates that the test molecule has a carcinogenic, metastatic, or tumorigenic effect.

The decrease of inhibition in the test cell culture can be compared to the level of mitochondrial trafficking or oxidative phosphorylation or cell respiration in untreated normal cell cultures. In another embodiment, the decrease of inhibition in the test cell culture can be compared to the level of mitochondrial trafficking or oxidative phosphorylation or cell respiration in untreated cancer/tumor cell cultures. In another embodiment, the decrease of inhibition in the test cell culture can be compared to the level of mitochondrial trafficking or oxidative phosphorylation or cell respiration in cancer/tumor cell cultures previously treated with a PI3K inhibitor or exposed to, or treated with some other cancer therapy. In another embodiment, the decrease of inhibition in the test cell culture can be compared to the level of mitochondrial trafficking or oxidative phosphorylation or cell respiration in cancer/tumor cell cultures established from refractory cancer cells or cultures established from known metastatic cancer cells, and the like.

The methodologies for conducting the screens, e.g., the assays and techniques for determine oxidative phosphorylation, cell respiration and mitochondrial movement, are selected from the techniques employed in the examples below or alternatively from among known techniques.

The following examples are provided for the purpose of illustration. These examples should be construed to encompass any and all variations that become evident as a result of the teaching provided herein.

### EXAMPLE 1

### Methods and Materials

2D Chemotaxis: Cells were treated with PI3K inhibitors for 48 h, and seeded in 2D chemotaxis chambers (Ibidi) in 10% FBS medium. After 6 h attachment, cells were washed and the reservoirs were filled with 0.1% BSA/RPMI, followed by gradient setup by addition of NIH3T3 conditioned medium. Video microscopy was performed over 8 h, with a time-lapse interval of 10 min At least 30 cells were tracked using the WimTaxis module (Wimasis), and the tracking data was exported into the Chemotaxis and Migration Tool v2.0 (Ibidi) for graphing and statistical testing. Experiments were repeated twice (n=3).

FA Dynamics: Cells growing in high optical quality 96 well μ-plates (Ibidi) were transduced with Talin-GFP BacMam virus (50 particles/cell) for 18 h and imaged with a 40× objective on a Nikon TE300 inverted time-lapse microscope equipped with a video system containing an Evolution QEi camera and a time-lapse video cassette recorder. The atmosphere was equilibrated to 37° C. and 5% CO₂ in an incubation chamber. Time lapse fluorescence microscopy of was carried out for the indicated times at 1 min/frame. Sequences were aligned in Image ProPlus7 and imported into ImageJ for further analysis. The initial and final frames were duplicated and assembled as composite images. FA complexes were manually counted and classified according to the presence in some or all of the time frames: decaying, newly formed, stable sliding (FA moves to a different position over time) and stable mature (merged areas). The rate of decay and assembly of FA complexes was calculated for each cells as the number of FA changing per h. At least 400 FA complexes from 10 cells were analyzed from 5 independent time lapses per condition.

Tumor cell invasion: Experiments were carried out essentially as described (41). Briefly, 8 μm PET transwell migration chambers (Corning) were coated with 150 μl of 80 μg/ml Matrigel (Becton Dickinson). Tumor cells were seeded in duplicates onto the coated transwell filters at a density of 1.25×10⁵ cells/well in media containing 2% FCS (FCIII, Hyclone) and media containing 20% FCS was placed in the lower chamber as chemoattractant. Cells were allowed to invade and adhere to the bottom of the plate stained in 0.5% crystal violet/methanol for 10 min, rinsed in tap water and analyzed by bright field microscopy. Digital images were batch imported into ImageJ, thresholded and analyzed with the Analyze particles function. For analysis of tumor cell invasion in 3D spheroids, tissue culture-treated 96-well plates were coated with 50 μl 1% DIFCO Agar Noble (Becton Dickinson). LN229 cells were seeded at 5,000 cells/well and allowed to form spheroids over 72 h. Spheroids were harvested, treated with PX-866 (0-10 □M) and placed in a collagen plug containing EMEM, FBS, L-Glutamine, sodium bicarbonate, and collagen type I (Gibco, 1.5 mg/ml). The collagen plug was allowed to set and 1 ml DMEM with 5% FBS was added to the top of the plug. Cell invasion was analyzed every 24 h and quantified using Image Pro Plus 7, as described⁴¹.

Statistical Analysis: Data were analyzed using either two-sided unpaired t test (for two group comparisons) or one way ANOVA test with Dunnett's multiple comparison post-test (for more than two group comparisons) using a GraphPad software package (Prism 6.0) for Windows. Data are expressed as mean±SD or mean±SEM of multiple independent experiments. A p value of <0.05 was considered as statistically significant.

### EXAMPLE 2

### P13K Therapy Reactivates Akt and Mammalian Target of Rapamycin Signaling

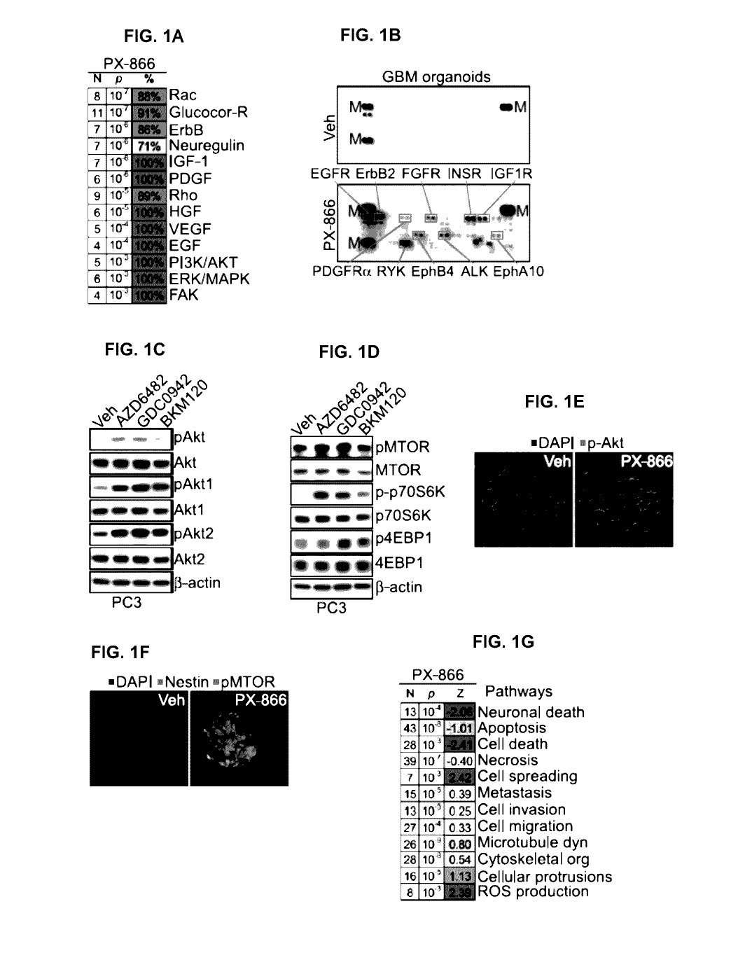

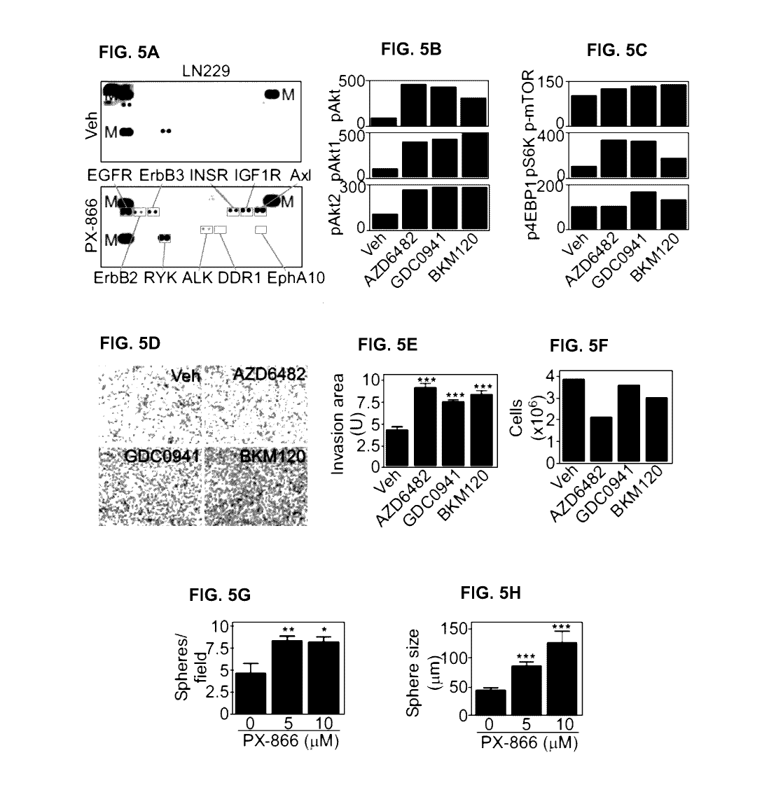

Treatment of patient-derived glioblastoma (GBM) organotypic culture5³ with PX-866, an irreversible pan-PI3K antagonist currently in the clinic⁴, caused transcriptional upregulation of multiple growth factor receptor pathways (FIG. 1A). This was associated with widespread phosphorylation, i.e. activation of the GBM kinome in primary organotypic cultures (FIG. 1B), as well as GBM LN229 cells (FIG. 5A). Consistent with previous observations⁸, structurally diverse small molecule PI3K antagonists induced robust (re)phosphorylation of Akt1 (S473) and Akt2 (S474) in tumor cells (FIGS. 1C and 5B), as well as phosphorylation of downstream mammalian target of rapamycin (mTOR) and its effectors, 70S6K and 4EBP 1 (FIGS. 1D and 5C). Similar results were obtained in primary 3D GBM neurospheres, where PI3K therapy strongly induced Akt (FIG. 1E) and mTOR (FIG. 1F) phosphorylation. By transcriptome analysis, PI3K antagonists upregulated two main gene networks of protection from apoptosis⁹ and increased cell motility (FIG. 1G) in treated tumors.

### EXAMPLE 2

### Increased Tumor Cell Motility Mediated by P13K Therapy

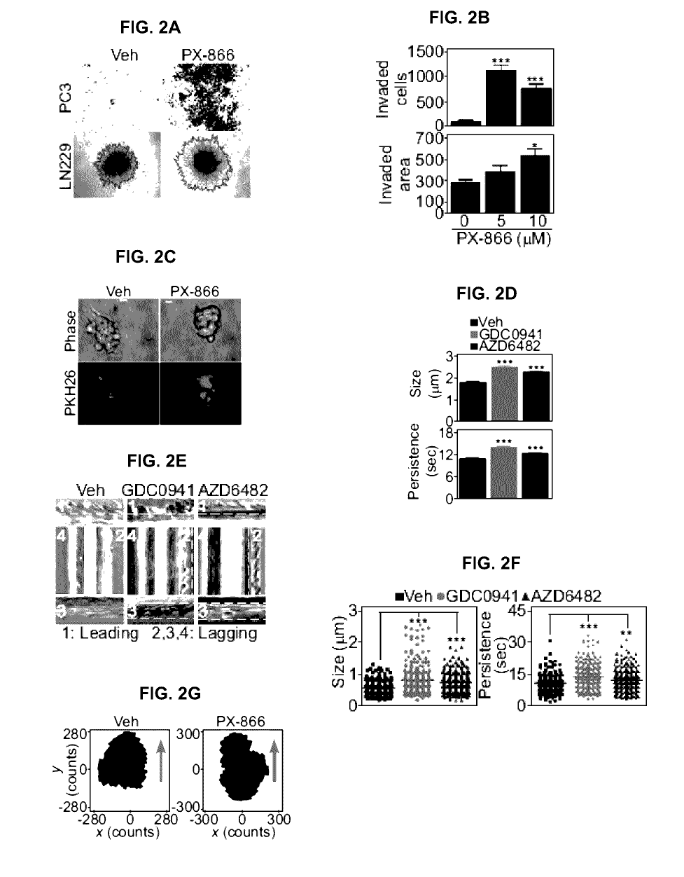

Consistent with these data, PI3K inhibitors vigorously stimulated tumor cell invasion across Matrigel-coated Transwell inserts (FIGS. 2A, 2B, 5D and 5E), and in 3D tumor spheroids (FIGS. 2A and 2B). Tumor cell proliferation was not significantly affected (FIG. 5F) (9). In addition, PI3K therapy dose-dependently increased the number and size of 3D GBM neurospheres (FIGS. 2C, 5G and 5H).

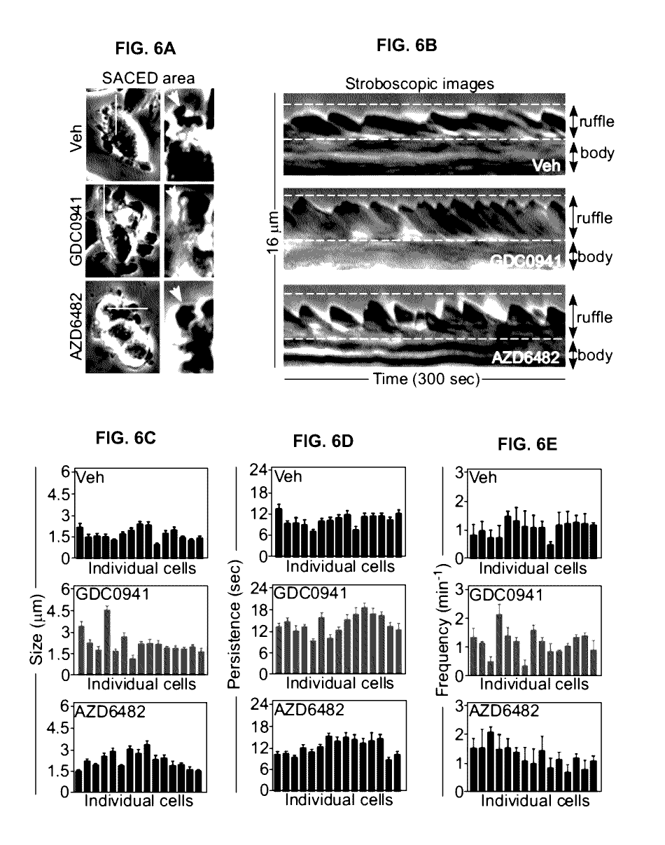

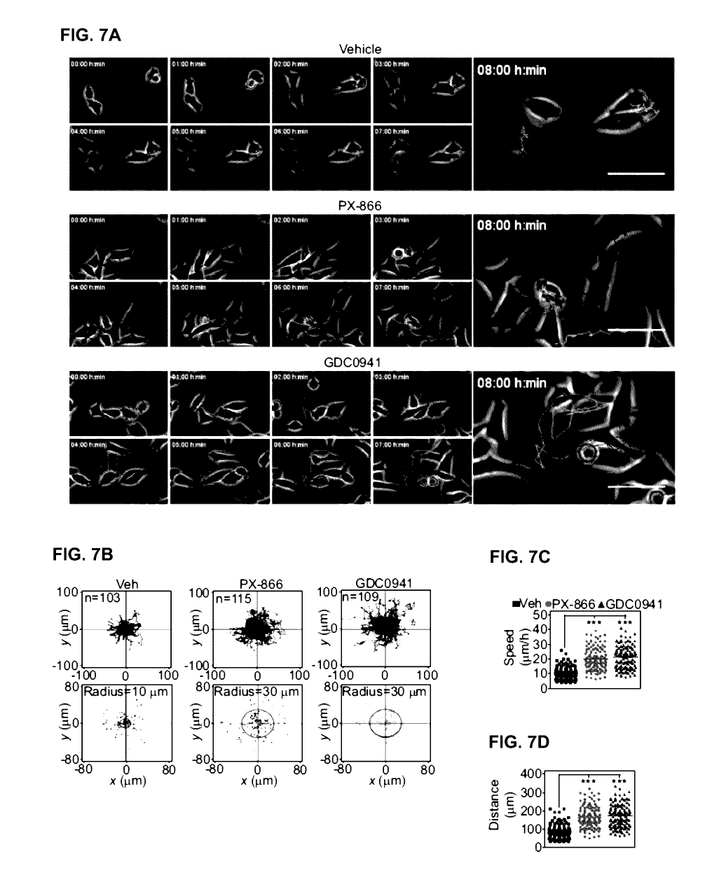

To understand the basis of this cell invasion response, we next quantified the dynamics of membrane lamellipodia, which are required for cell motility, by single cell stroboscopic microscopy¹⁴ (FIG. 6A). PI3K antagonists strongly stimulated lamellipodia dynamics (FIG. 6B), increasing the size (FIGS. 2D, top and 6C), and time of persistence (FIGS. 2D, bottom and 6D) of membrane ruffles, compared with control cultures. Ruffle frequency was not affected (FIG. 6E). In addition, PI3K therapy changed the topography of membrane ruffles in tumor cells, with appearance of dynamic ruffles at lagging areas of the plasma membrane (FIG. 2E), potentially associated with random cell motility¹⁵. These lateral ruffles were larger and persisted for longer time in response to PI3K therapy, compared with untreated cells (FIG. 2F), where membrane ruffles were instead polarized at the leading edge of migration (FIG. 2E). Consistent with these findings, PI3K antagonists strongly stimulated 2D tumor chemotaxis (FIG. 7A), extending the radius of cell migration (FIG. 7B), and promoting random, as opposed to directional cell movements (FIG. 2G). Tumor cell movement in response to PI3K therapy proceeded at faster speed (FIG. 7C), and for longer distances (FIG. 7D), compared with untreated cultures.

### EXAMPLE 3

### Mitochondrial Repositioning to the Cortical Cytoskeleton Supports Adaptive Tumor Cell Invasion

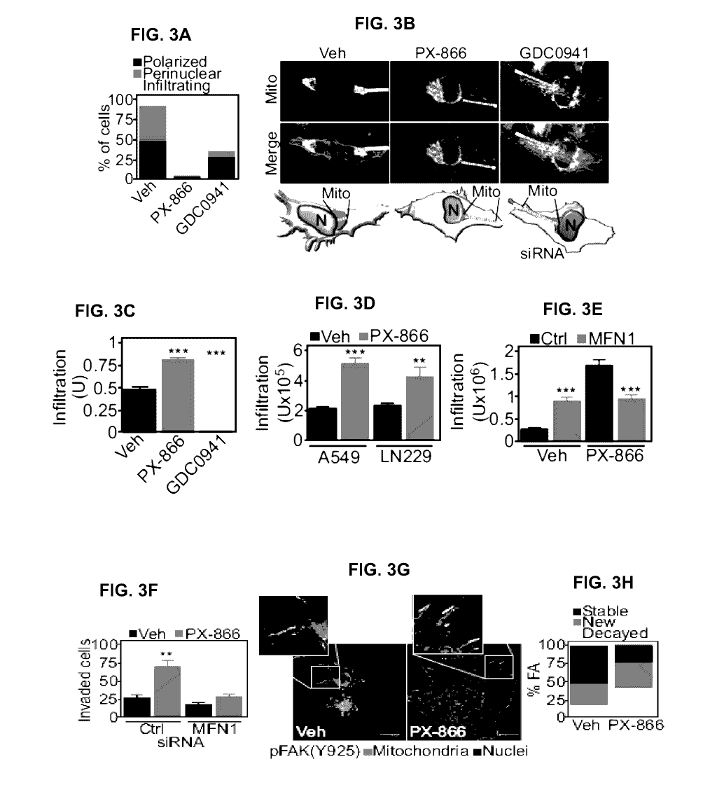

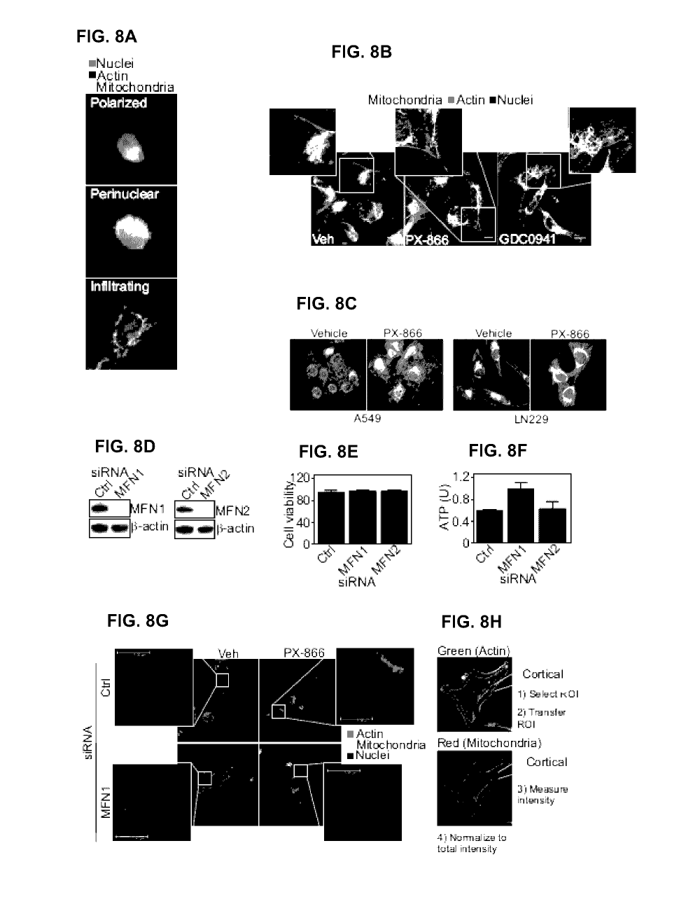

When analyzed by fluorescence microscopy, PI3K therapy induced profound changes in the morphology and distribution of mitochondria. Whereas untreated cells exhibited mitochondria that were polarized and mostly clustered around the nucleus (FIGS. 8A and B), PI3K inhibitors caused the appearance of elongated mitochondria (FIG. 8A) that “infiltrated” the cortical cytoskeleton of tumor cells, localizing in proximity of membrane protrusions implicated in cell motility (FIG. 3A-C and FIG. 8B). This was a general response of heterogeneous tumor cell types, as lung adenocarcinoma A549 or glioblastoma LN229 cells, comparably repositioned mitochondria to the cortical cytoskeleton in response to PI3K therapy (FIG. 3D and FIG. 8C). Mitochondria are highly dynamic organelles, regulated by cycles of fusion and fission¹⁶. Small interfering RNA (siRNA) knockdown of effectors of mitochondrial fusion, mitofusin (MFN)-1 or MFN-2 (FIG. 8D) did not affect cell viability (FIG. 8E) or ATP production (FIG. 8F) in tumor cells. Under these conditions, MFN 1 silencing suppressed mitochondrial trafficking to the cortical cytoskeleton (FIG. 3E and FIG. 8G and H), as well as tumor cell invasion (FIG. 3F) induced by PI3K therapy. The combination of MFN2 knockdown plus PI3K inhibition induced extensive loss of cell viability (MFN1 siRNA+PX-866, 0.27±0.005×106 cells; MFN2 siRNA+PX-866, 0.016±0.013×106 cells, p=0.0047), thus preventing additional studies of mitochondrial relocalization or tumor cell invasion.

### EXAMPLE 4

### Requirements of Mitochondrial Regulation of Tumor Cell Invasion

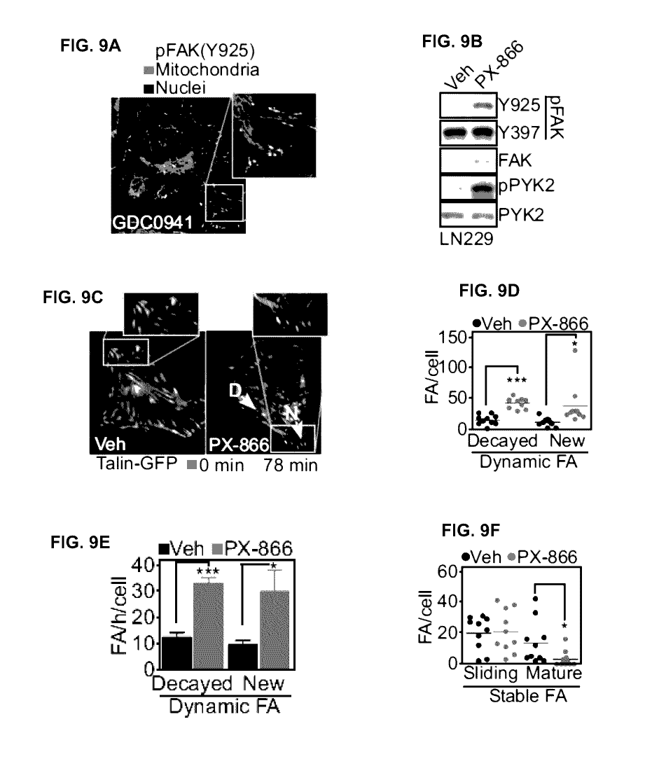

A prerequisite of cell movements is the timely assembly/disassembly of focal adhesion (FA) complexes¹⁴, and a role of mitochondrial trafficking in this process was next investigated. Mitochondria repositioned to the cortical cytoskeleton in response to PI3K antagonists co-localized with phosphorylated (Y925) Focal Adhesion Kinase (FAK) (FIG. 3G and FIG. 9A), which regulates FA dynamics¹⁷. This was associated with increased FAK phosphorylation (Y925), compared with control cultures (FIG. 9B). By time lapse videomicroscopy (FIG. 9C), PI3K therapy profoundly affected FA dynamics (FIG. 3H), increasing both the assembly and decay of FA complexes (FIG. 9D), and their turnover rate (FIG. 9E). In contrast, PI3K inhibition reduced the number of stable FA complexes (FIG. 9F).

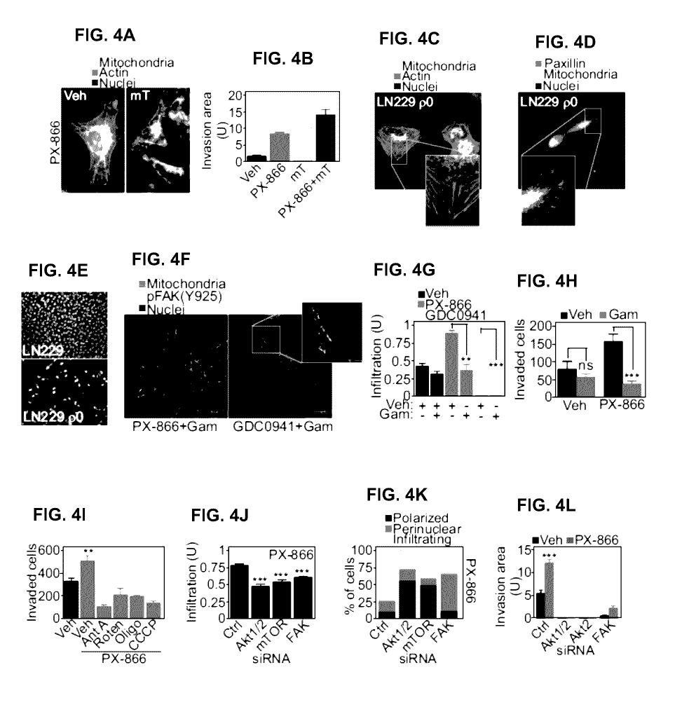

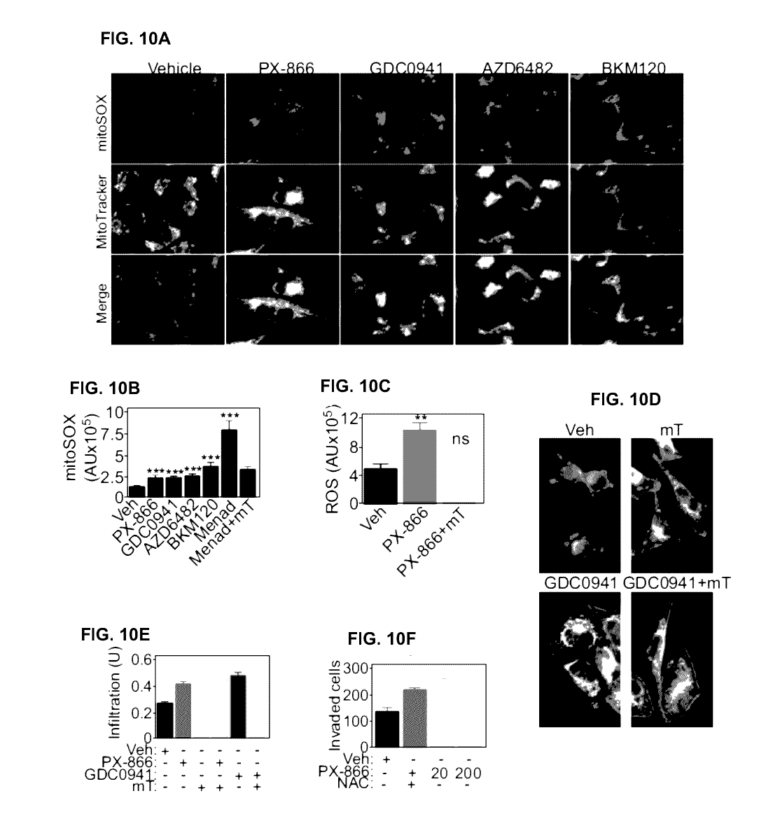

Mitochondria are a primary source of reactive oxygen species (ROS), and these moieties have been implicated in tumor cell motility. PI3K antagonists increased the production of mitochondrial superoxide in tumor cells, compared with untreated cultures (FIGS. 10A and B), and this response was abolished by a mitochondrial-targeted ROS scavenger, mitoTEMPO (FIG. 10C). In contrast, ROS scavenging with mitoTEMPO did not affect mitochondrial repositioning to the cortical cytoskeleton (FIG. 4A and FIG. 10D and E) or tumor cell invasion (FIG. 4B) mediated by PI3K inhibitors. Increasing concentrations of the pan-antioxidant N-acetyl cysteine (NAC) also had no effect on PI3K therapy-mediated tumor cell invasion (FIG. 10F). The increase in basal cell motility in the presence of antioxidants may reflect release of ROS-regulated inhibitory mechanisms of mitochondrial trafficking.

### EXAMPLE 5

### Role of Bioenergetics in Mitochondrial Trafficking and Tumor Cell Invasion

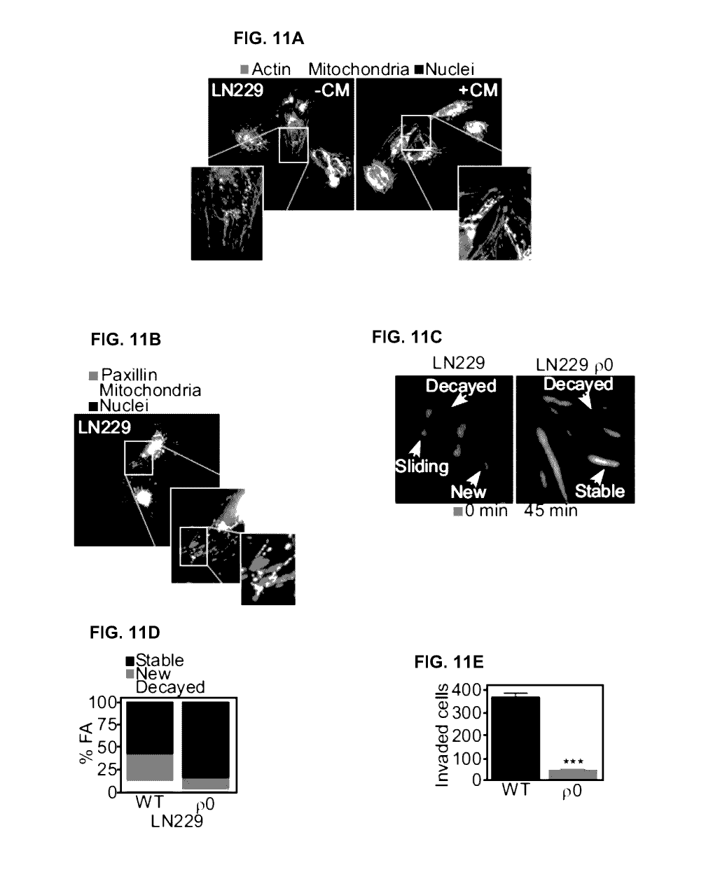

Next, we asked whether mitochondrial bioenergetics was important for this pathway, and generated LN229 cells devoid of oxidative phosphorylation (ρ0 cells). Chemoattractant stimulation of respiration-competent LN229 cells induced repositioning of mitochondria to the cortical cytoskeleton (FIG. 11A) that co-localized with paxillin+ FA complexes (FIG. 11B). In contrast, respiration-deficient LN229 ρ0 cells failed to reposition mitochondria to the cortical cytoskeleton (FIG. 4C). This absence of mitochondria proximal to FA complexes (FIG. 4D) was associated with loss of FA dynamics (FIGS. 11C and 11D), and suppression of tumor cell invasion across Matrigel-containing inserts (FIG. 4E and FIG. 11E).

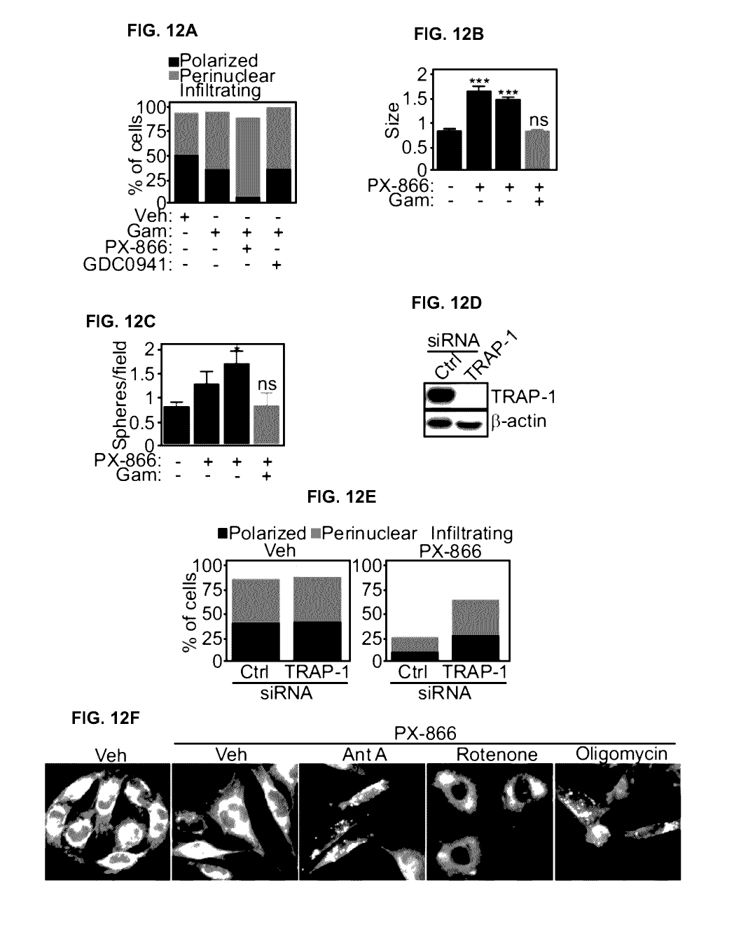

As an independent approach, we treated tumor cells with Gamitrinib, a mitochondrial-targeted small molecule Hsp90 inhibitor that induces misfolding and degradation of oxidative phosphorylation Complex II subunit, SDHB¹⁸. Non-toxic concentrations of Gamitrinib abolished the trafficking of mitochondria to pFAK-containing FA complexes in response to PI3K antagonists (FIG. 4F and G), and preserved a polarized and perinuclear mitochondrial distribution (FIG. 12A). Consistent with these findings, Gamitrinib abolished the increase in tumor cell invasion (FIG. 4H), and the expansion of primary GBM neurospheres (FIGS. 12B and 12C) mediated by PI3K antagonists. To validate these findings, we next silenced the expression of TRAP-1 (FIG. 12D), a mitochondrial Hsp90-like chaperone targeted by Gamitrinib and implicated in Complex II stability (18). TRAP-1 silencing in vehicle-treated cells did not affect mitochondrial localization (FIG. 12E, left). In contrast, knockdown of TRAP-1 abolished mitochondrial trafficking to the cortical cytoskeleton in the presence of PI3K antagonists, increasing the fraction of polarized and perinuclear organelles in these cells (FIG. 12E, right). Finally, treatment with small molecule inhibitors of mitochondrial Complex I (Rotenone), Complex III (Antimycin A), Complex V (Oligomycin) or a mitochondrial uncoupler (carbonyl cyanide m-chlorophenyl hydrazine; CCCP), inhibited mitochondrial repositioning to the cortical cytoskeleton (FIG. 12F) and tumor cell invasion (FIG. 41) in the presence of PI3K therapy.

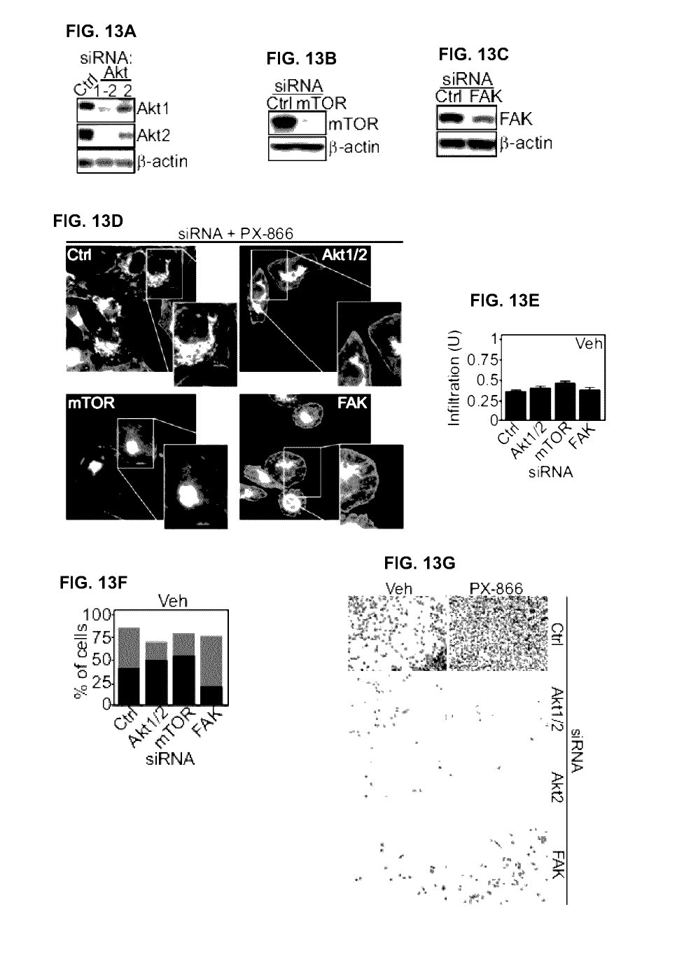

To begin elucidating the signaling requirements of adaptive mitochondrial trafficking and tumor cell invasion, we next targeted the PI3K-Akt-mTOR axis, which becomes reactivated in response to PI3K therapy^{8, 9}. Knockdown of Akt1 or Akt2 (FIG. 13A), mTOR (FIG. 13B) or FAK (FIG. 13C) independently prevented the repositioning of mitochondria to the cortical cytoskeleton (FIG. 13D, FIG. 4J and K), and suppressed tumor cell invasion (FIG. 13G and FIG. 4L) induced by PI3K antagonists. In contrast, knockdown of these molecules in the absence of PI3K inhibition had no effect on mitochondrial trafficking (FIG. 13E), or organelle morphology (FIG. 13F).

Antibodies and reagents: Antibodies to pan Ser473/474-phosphorylated Akt 1/2 (Cell Signaling), pan Akt (Cell Signaling), Ser473-phosphorylated Akt1 (Cell Signaling), Akt1 (cell Signaling), Ser474-phosphorylated Akt2 (Cell Signaling), Akt2 (Cell Signaling), Ser2448-phosphorylated MTOR (Cell Signaling), MTOR (Cell Signaling), Thr389-phosphorylated p70S6K (Cell Signaling), p70S6K (Cell Signaling), Thr37/46-phosphorylated 4EBP1 (Cell Signaling), MFN1 (Abeam), MFN2 (Abeam), Tyr397-phosphorylated FAK (Cell Signaling), Tyr925-phosphorylated FAK (Cell Signaling), FAK (Cell Signaling), Tyr402-phosphorylated PYK2 (Cell Signaling), PYK2 (Cell Signaling), FIP200 (Cell Signaling), ULK1 (Santa Cruz), TRAP1 (BD), and β-actin (Sigma-Aldrich) were used for Western blotting. Antibodies to Tyr925-phosphorylated FAK (Cell Signaling) and paxillin (Upstate) were used for immunofluorescence. The PI3K inhibitors (AZD6482, GCD0941, BKM120) were purchased from Selleckchem, PX-866 was from LC laboratories. mitoTEMPO was purchansed from Santa Cruz. N-acetyl cysteine (NAC) and carbonyl cyanide m-chlorophenyl hydrazine (CCCP) were from Sigma-Aldrich. Antimycin A, Oligomycin, and Rotenone were obtained from Abcam Biochemicals. All chemicals were of the highest purity commercially available. The complete chemical synthesis, HPLC profile, and mass spectrometry of mitochondrial-targeted Hsp90 antagonist, Gamitrinib has been reported⁴². The Gamitrinib variant containing triphenylphosphonium as a mitochondrial-targeting moiety was used throughout this study. Phalloidin Alexa488, Mitotracker® Red CMXRos, MitoSOX™ Red mitochondrial superoxide indicator, CellLight® Talin-GFP BacMam 2.0 and secondary antibodies for immunofluorescence were from Molecular Probes.

Cell culture and drug treatment: Human GBM LN229, prostate adenocarcinoma PC3, lung carcinoma A549, and mouse fibroblasts NIH3T3 cells were obtained from the American Type Culture Collection (ATCC, Manassas, Va.), and maintained in culture according to the supplier's specifications. Conditioned media was prepared from exponentially growing cultures of NIH3T3 cells as previously described⁴¹, in DMEM supplemented with 4.5 g/l D-glucose, sodium pyruvate, 10 mM HEPES and 10% FBS for 48 h. Where indicated, starvation was carried out in DMEM without glucose for 24 h. PI3K inhibitors were dissolved in DMSO at 10 mM and added to the culture media for 48 h at the following concentrations: AZD6482 at 10 μM, GCD0941 at 2 μM, BKM120 at 0.4 μM and PX-866 at 5 μM. mitoTEMPO was added to the culture media at 50-100 μM, NAC at 20 μM, Antimycin A at 30 μM, Oligomycin at 2.5 μM, Rotenone at 4 μM and CCCP at 12.5 μM. For invasions, the antioxidants, ROS scavengers, and ETC inhibitors were added to the top and bottom of the chambers at the moment of cell seeding.

Generation of LN229 ρ0 cell line: Mitochondrial DNA-deficient LN229 cells were generated by a 3 week-culture in the presence of ethidium bromide (50 ng/ml) in medium supplemented with 1 mM sodium pyruvate and 50 μg/mluridine. mtDNA depletion was confirmed by PCR to be >90%. Briefly, total DNA (both mitochondrial and genomic) was extracted from LN229 or LN229 ρ0 cultures using the QIAamp DNA Mini Kit (Qiagen). Real-time PCR was carried out in an Applied Biosystems Inc. (ABI) using the SYBR-Green PCR Master Mix (Qiagen). Primers for two mtDNA markers (□-globin and 18S rRNA) was used Amplifications were performed in 25 μl reactions comprising of 15 ng total DNA, 1× SYBR-Green PCR Master Mix (Qiagen) with 3.5 mM MgCl₂, and 0.3 μM each primer. Triplicate reactions were performed for each marker in a 96-well plate.

Patients: Fresh, patient-derived tissues obtained from surgical resections of grade IV glioblastoma (GBM, 7 cases) were used in this study. Informed consent was obtained from all patients, and the study was approved by an Institutional Review Board at the Fondazione IRCCS Ca′ Granda hospital (Milan, Italy). The clinicopathological characteristics of the patient series used are presented in Table 1 below.

TABLE 1

| Patient ID | Sex | Age (yr) | Grade | Ki-67% | MGMT % | | LOH |
|---|---|---|---|---|---|---|---|
| GBM1 | M | 66 | IV | 50 | 57 | (M) | No |
| GBM2 | F | 64 | IV | 30 | 54 | (M) | 1p/19q |
| GBM3 | F | 78 | IV | 35 | 62 | (M) | No |
| GBM4 | M | 64 | IV | 50 | 4 | (UM) | No |
| GBM5 | F | 63 | IV | 35 | 9 | (UM) | 19q |
| GBM6 | M | 53 | IV | 15 | 3 | (UM) | No |
| GBM7 | M | 36 | IV | 80 | 24 | (M) | No |

The clinicopathological features of GBM patients used for organotypic glioma cultures (GBM 1 and 2) or neurosphere derivation (GBM 3-7) are indicated. F, female; M, male. Age (years) at diagnosis is indicated. Grade was estimated according to the fourth edition of the World Health Organization (WHO) classification of tumors of the central nervous system (2007). The Ki-67 labeling index was considered in tumor hotspots as an indicator of glioma proliferation. The percentage of MGMT promoter methylation (by pyrosequencing analysis) and the resulting status (methylated if ≧20%, or unmethylated) is provided. M, methylated; UM, unmethylated. Loss of heterozygosity (LOH) at 1p/19q was analyzed.

Organotypic cultures: Short-term organotypic cultures from primary patient samples were established as described¹³. Briefly, precise thick tissue slices (300 μm) were obtained by vibratome serial cutting (VT1200, Leica Microsystems, Milan, Italy), and cultured in six-well plates on organotypic inserts (Millicell PICM ORG, Merck Millipore) for up to 48 h in 1 ml of Ham-F12 complete medium supplemented with vehicle (DMSO, 2.5 μl) or pan-PI3K inhibitor PX-886 (10 μM). At the end of the experiment, one tissue slice per condition was formalin-fixed and paraffin-embedded, and further processed for morphological and immunohistochemical analysis. An additional tissue slice was embedded in OCT, and snap-frozen for molecular or immunofluorescence studies.

GBM neurospheres: Fresh GBM specimens collected from consenting patients (Division of Neurosurgery, Fondazione IRCCS Ca′ Granda, Milan, Italy) were immediately processed for the isolation of stem cell-containing neurospheres. Briefly, tissues were enzymatically processed with the Tumor Dissociation Kit (Miltenyi Biotech) at 37° C. in combination with the gentleMACS™ Dissociators (Miltenyi Biotech) until achievement of single cell suspension. Then, dissociated GBM cells were grown as spheroid aggregates (neurospheres) as previously described⁴³ and using the NeuroCult NS-A Proliferation media (StemCell Technologies) supplemented with 20 ng/ml EGF and 10 ng/ml bFGF (Stem Cell Technologies) according to manufacturer's protocol. When pharmacological treatment was performed, neurospheres derived from five GBM patients were mechanically dissociated, stained with PKH26 (Sigma Aldrich) as described⁴⁴, and plated at a density of 1000 cells per well in 24-well plates. The intended use of the non-toxic dye PKH26 was for live cell identification and staining. Twenty-four h later, cells were treated with 5 or 10 μM of PX-886 in the presence of absence of Gamitrinib (10 μM) and monitored for neurosphere formation for up to three days. During this interval, neurospheres were imaged every 24 h and their size (diameter) and number was assessed by direct counting using ImageJ software. PKH26 was monitored and recorded using the TRITC fluorescent filter. Experiments were performed in duplicates. For immunofluorescence experiments, control or PX-866 (5-10 □M)-treated neurospheres were fixed in 4% PFA for 15 min and placed on charged glasses using a cytospin. Samples were further incubated with primary antibodies against phosphorylated Akt (Ser473; Cell Signaling), phosphorylated mTOR (Ser2448; Cell Signaling), or Nestin (clone 196908, R&D System) and mounted in a media containing DAPI (Abbott).

Plasmids and mutagenesis. The following plasmids were obtained from Addgene: FIP200 (#24303) and ULK1 (#27629). A Ser757→Ala ULK1 mutant was generated using QuikChange II XL Site-Directed Mutagenesis Kit (Agilent Technologies) and confirmed by DNA sequencing. Cells were transfected with 2 μg of pDNA plus 4 μl X-treme gene HP (Roche) for 24 h, washed and subject to the treatments indicated.

Small interfering RNA (siRNA). Knockdown experiments were carried out as described⁴¹. The following siRNA were used: control, non-targeting small interfering RNA (siRNA) pool (Dharmacon, D-001810) or specific siRNA pools targeting Akt1/2 (Santa Cruz, sc-43609), Akt2 (Santa Cruz, sc-29197), MTOR (Dharmacon, L-003008), FAK (Santa Cruz, sc-29310), TRAP1 (Dharmacon, L-010104), MFN1 (Santa Cruz, sc-43927), MFN2 (Santa Cruz, sc-43928). siRNA were transfected at 10-30 nM concentrations in the presence of Lipofectamine RNAiMAX (Invitrogen) at a 1:1 ratio (vol siRNA 20 μM:vol Lipofectamine RNAiMAX). Cells were incubated for 48 h, validated for target protein knockdown by Western blotting, and processed for subsequent experiments. PC3 cells stably expressing shRNA targeting FIP200 were generated by infection with lentiviral particles, followed by 2 weeks of selection with puromycin at 2 μg/ml. Two shRNA sequences were used for targeting FIP200: TRCN000013523 and TRCN0000350426 (Sigma-Aldrich). An empty pLKO-based lentivirus was used as control.

Kinome profiling and bioinformatics analysis. Transcriptional changes in the human kinome following PI3K inhibition were analyzed using OpenArray real-time PCR amplification. GBM organotypic cultures were treated with vehicle (DMSO) or PX-866 (10 μM) for 48 h. Total RNA was extracted from each sample, reverse-transcribed and the derived cDNA was hybridized with TaqMan® Array Human Protein Kinase Pathways (cat. n. 4414076). Ct values from the experiment were exported from BioTrove OpenArray Real-Time qPCR Analysis Software v1.0.4. The ΔΔCt method was used to calculate fold changes between sample pairs with endogenous control Ct value calculated as average among 15 endogenous controls on the array: ALAS1, B2M, CASC3, G6PD, GAPDH, GUSB, HMBS, HPRT1, IPO8, POLR2A, PPIA, RPLP0, TFRC, UBE2D2, and YWHAZ. The p values for differential expression were calculated using fold changes of 15 endogenous controls as a null distribution. FDR values were estimated using Benjamini-Hochberg correction for multiple testing and results with FDR<20% were used for enrichment analysis with Ingenuity Pathway Analysis software (Ingenuity Systems, Richmond, Calif.) to identify a list of pathways and functions significantly overrepresented among the significantly changed genes.

Protein analysis. For Western blotting, protein lysates were prepared in Triton X-100 lysis buffer (20 mM TrisClH pH 8.00, 137 mM NaCl, 1% NP-40, 10% glycerol) containing EDTA-free Protease Inhibitor Cocktail (Roche) and Phosphatase Inhibitor Cocktail 2 and 3 (Sigma-Aldrich), sonicated and precleared by centrifugation at 14,000 rpm for 10 min, at 4° C. Equal amounts of protein lysates were separated by SDS gel electrophoresis, transferred to PVDF membranes, blocked in 5% low fat milk/TBST and incubated with primary antibodies of various specificities diluted 1:1000 in 5% BSA/TBST, overnight at 4° C. After washing in TBST, membranes were incubated with secondary antibodies conjugated to HRP (1:1000-1:5000 dilution in 5% BSA/TBST) for 1 h at room temperature, washed with TBST and protein bands were visualized by enhanced chemiluminescence.

Membrane ruffling dynamics in live cells. Quantification of membrane ruffle dynamics in live cells was carried out as described previously⁴¹. Briefly, 3-5×10⁴ cells were grown on high optical quality 96 well m-plates (Ibidi) and imaged with a 40× objective on a Nikon TE300 inverted time-lapse microscope equipped with a video system containing an Evolution QEi camera and a time-lapse video cassette recorder. The atmosphere was equilibrated to 37° C. and 5% CO₂ in an incubation chamber. Phase contrast images were captured at 0.5s intervals for 250 seconds (500 frames) and merged into sequence files using ImagePro Plus 7. To monitor dynamics of a particular region by Stroboscopic Analysis of Cell Dynamics (SACED)⁴⁵, the sequence files were imported into Image J, and a particular region of 16.2 mm×0.162 mm (”SACED line“) was selected, duplicated and montaged in sequence to display the region over time in a stroboscopic image. This process was repeated to get 4 SACED lines and therefore 4 stroboscopic images per cell (n=15), and structures such as protruding lamellipodia and ruffles were manually labeled and the length and angle obtained were used to calculate the distance and time of each event by trigonometry. For each cell, the frequency of ruffles per min, the ruffling retraction speed (microns/min), the ruffle migration distance (micrometers) and persistence (seconds) were calculated. Mean values were calculated from at least 15 cells from 3-4 independent time lapses, as described⁴¹.

Detection of mitochondrial superoxide in live cells. The production of superoxide by mitochondria was visualized in fluorescence microscopy using the MitoSOX™ Red reagent. Briefly, 1.5×10⁴ cells were grown on high optical quality 8 well μ-slides (Ibidi) and imaged with a 40× objective on a Nikon TE300 inverted time-lapse microscope equipped with a video system containing an Evolution QEi camera and a time-lapse video cassette recorder. The atmosphere was equilibrated to 37° C. and 5% CO₂ in an incubation chamber. Phase contrast and TRITC (TRITC filter cube, excitation wavelength: 532-554 nm, emission wavelength: 570-613 nm) images were captured. To quantitate superoxide levels, files were imported into Image J, masks were manually created around the periphery of the cell based on the Phase image and subsequently applied to the TRITC channel to measure intensity. A minimum of 100 cells were analyzed in each independent experiment to obtain mean values.

Mitochondrial localization. After the indicated transfections or treatments, cells were stained with MitoTracker Red CMXRos (100 nM, 2 h) at 37° C. After fixation with 4% formalin in PBS for 15 min at room temperature, cells were permeabilized with 0.1% Triton X-100/PBS for 5 min, washed, and incubated 30 min with 1% BSA/0.3M glycine/PBS. F-actin was stained with phalloidin Alexa488 (1:1000 dilution) for 30 min at room temperature. Slides were washed and mounted in DAPI-containing Prolong Gold mounting medium (Invitrogen). At least 7 random fields were analyzed by fluorescence microscopy in a Nikon E600 microscope. Composite images were analyzed in Image J, for each cell two lamellipodia were measured to calculate infiltration index, defined as the radial distance from nucleus to furthest mitochondrial string, normalized to the radial distance from nucleus to cell border. In parallel, all cells on the 7 fields were scored into 3 categories, according to gross localization of mitochondria: polarized, pen-nuclear, and infiltrating. For cortical mitochondria quantification, a mask was manually created around the periphery of the cell based on the F-actin channel and subsequently applied to the mitochondria channel to measure intensity at the cortical region (FIG. 8H). The intensity was normalized to total mitochondria intensity per cell and background subtracted. A minimum of 20 cells were analyzed in each independent experiment to obtain mean values. For high resolution imaging of mitochondria in MFN1-depleted cells, slides were sequentially scanned for red, green, and blue fluorescence by a Leica SP5 confocal microscope with a 63× objective and a 20× digital zoom. Full cell z stacks (about 5-8 μm) were postprocessed in Leica Application Suite Advanced Fluorescence (LAS AF) to reduce noise by applying a median filter (two iterations, radius 3) and used to generate max projections.

Focal adhesion (FA) complexes and mitochondria dual labeling. Coverslides were coated with fibronectin (10 μg/ml in PBS) overnight at 4° C., rinsed in PBS and equilibrated at 37° C. with 500 μl of 10% FBS/RPMI. Cells were treated as indicated, tripsinized, washed with 10% FBS/RPMI and plated on fibronectin-coated slides, in the presence of Mitotracker Red CMXRos (100 nM). After 5 h, cells were fixed with 10% formalin in PBS for 15 min at room temperature, permeabilized with 0.1% Triton X-100/PBS for 5 min, washed, and FAs were labeled with primary antibodies diluted in 1% BSA/0.3M glycine/PBS, overnight at 4° C. Either one of the following antibodies were used: rabbit anti-pY925-FAK (1:50, Cell Signaling), mouse anti-Paxillin (1:100, Upstate). Next, slides were washed, incubated with secondary antibodies (1:500, Alexa Fluor 488 conjugated) for 1 h at room temperature, washed and mounted in DAPI-containing Prolong Gold mounting medium (Invitrogen). Slides were sequentially scanned for red, green and blue fluorescence in a Leica SP5 confocal microscope with a 100× objective. Libraries were imported into LASLite and a 3 step z-stack centered at the plane of focal adhesions (±0.5 μm) were used to generate 3D max projections.

Each and every patent, patent application, and publication, including priority provisional application No. 62/185,370, and including websites cited throughout the disclosure, is expressly incorporated herein by reference in its entirety. While this invention has been disclosed with reference to specific embodiments, it is apparent that other embodiments and variations of this invention are devised by others skilled in the art without departing from the true spirit and scope of the invention. The appended claims include such embodiments and equivalent variations.

### REFERENCES

1. Engelman J A, Luo J, & Cantley LC (2006) The evolution of phosphatidylinositol 3-kinases as regulators of growth and metabolism. Nat Rev Genet 7(8):606-619.
2. Manning B D & Cantley L C (2007) AKT/PKB signaling: navigating downstream. Cell 129(7):1261-1274.
3. Vivanco I & Sawyers C L (2002) The phosphatidylinositol 3-Kinase AKT pathway in human cancer. Nat Rev Cancer 2(7):489-501.
4. Rodon J, Dienstmann R, Serra V, & Tabernero J (2013) Development of PI3K inhibitors: lessons learned from early clinical trials. Nat Rev Clin Oncol 10(3):143-153.
5. Fruman D A & Rommel C (2014) PI3K and cancer: lessons, challenges and opportunities. Nat Rev Drug Discov 13(2):140-156.
6. Janne P A, Gray N, & Settleman J (2009) Factors underlying sensitivity of cancers to small-molecule kinase inhibitors. Nat Rev Drug Discov 8(9):709-723.
7. Cohen A A, et al. (2008) Dynamic proteomics of individual cancer cells in response to a drug. Science 322(5907):1511-1516.
8. Chakrabarty A, Sanchez V, Kuba M G, Rinehart C, & Arteaga C L (2012) Feedback upregulation of HER3 (ErbB3) expression and activity attenuates antitumor effect of PI3K inhibitors. Proc Natl Acad Sci USA 109(8):2718-2723.
9. Ghosh J C, et al. (2015) Adaptive mitochondrial reprogramming and resistance to PI3K therapy. J Natl Cancer Inst In press.
10. Ward P S & Thompson C B (2012) Metabolic reprogramming: a cancer hallmark even warburg did not anticipate. Cancer Cell 21(3):297-308.
11. Wallace D C (2012) Mitochondria and cancer. Nat Rev Cancer 12(10):685-698.
12. Frezza C, et al. (2011) Haem oxygenase is synthetically lethal with the tumour suppressor fumarate hydratase. Nature 477(7363):225-228.
13. Vaira V, et al. (2010) Preclinical model of organotypic culture for pharmacodynamic profiling of human tumors. Proc Natl Acad Sci USA 107(18):8352-8356.
14. Roussos E T, Condeelis J S, & Patsialou A (2011) Chemotaxis in cancer. Nat Rev Cancer 11(8):573-587.
15. Petrie R J, Doyle A D, & Yamada K M (2009) Random versus directionally persistent cell migration. Nat Rev Mol Cell Biol 10(8):538-549.
16. Youle R J & van der Bliek A M (2012) Mitochondrial fission, fusion, and stress. Science 337(6098):1062-1065.
17. Sulzmaier F J, Jean C, & Schlaepfer D D (2014) FAK in cancer: mechanistic findings and clinical applications. Nat Rev Cancer 14(9):598-610.
18. Chae Y C, et al. (2013) Landscape of the mitochondrial Hsp90 metabolome in tumours. Nat Commun 4:2139.
19. Janiszewska M, et al. (2012) Imp2 controls oxidative phosphorylation and is crucial for preserving glioblastoma cancer stem cells. Genes Dev 26(17):1926-1944.
20. Viale A, et al. (2014) Oncogene ablation-resistant pancreatic cancer cells depend on mitochondrial function. Nature 514(7524):628-632.
21. Haq R, et al. (2013) Oncogenic BRAF regulates oxidative metabolism via PGC1alpha and MITF. Cancer Cell 23(3):302-315.
22. LeBleu V S, et al. (2014) PGC-lalpha mediates mitochondrial biogenesis and oxidative phosphorylation in cancer cells to promote metastasis. Nat Cell Biol 16(10):992-1003.
23. Shiraishi T, et al. (2014) Glycolysis is the primary bioenergetic pathway for cell motility and cytoskeletal remodeling in human prostate and breast cancer cells. Oncotarget.
24. Porporato P E, et al. (2014) A mitochondrial switch promotes tumor metastasis. Cell Rep 8(3):754-766.
25. Olgun A & Akman S (2007) Mitochondrial DNA-deficient models and aging. Ann NY Acad Sci 1100:241-245.
26. De Bock K, et al. (2013) Role of PFKFB3-driven glycolysis in vessel sprouting. Cell 154(3):651-663.
27. Lee C W & Peng H B (2006) Mitochondrial clustering at the vertebrate neuromuscular junction during presynaptic differentiation. J Neurobiol 66(6):522-536.
28. Saxton W M & Hollenbeck P J (2012) The axonal transport of mitochondria. J Cell Sci 125(Pt 9):2095-2104.
29. Birsa N, Norkett R, Higgs N, Lopez-Domenech G, & Kittler J T (2013) Mitochondrial trafficking in neurons and the role of the Miro family of GTPase proteins. Biochem Soc Trans 41(6):1525-1531.
30. Zhao J, et al. (2013) Mitochondrial dynamics regulates migration and invasion of breast cancer cells. Oncogene 32(40):4814-4824.
31. Morlino G, et al. (2014) Miro-1 links mitochondria and microtubule Dynein motors to control lymphocyte migration and polarity. Mol Cell Biol 34(8):1412-1426.
32. Desai SP, Bhatia SN, Toner M, & Irimia D (2013) Mitochondrial localization and the persistent migration of epithelial cancer cells. Biophys J 104(9):2077-2088.
33. Kolsch V, Charest P G, & Firtel R A (2008) The regulation of cell motility and chemotaxis by phospholipid signaling. J Cell Sci 121(Pt 5):551-559.
34. Weiger M C, et al. (2009) Spontaneous phosphoinositide 3-kinase signaling dynamics drive spreading and random migration of fibroblasts. J Cell Sci 122(Pt 3):313-323.
35. Kraynov V S, et al. (2000) Localized Rac activation dynamics visualized in living cells. Science 290(5490):333-337.
36. Fabry B, Klemm A H, Kienle S, Schaffer T E, & Goldmann W H (2011) Focal adhesion kinase stabilizes the cytoskeleton. Biophys J 101(9):2131-2138.
37. Pennacchietti S, et al. (2003) Hypoxia promotes invasive growth by transcriptional activation of the met protooncogene. Cancer Cell 3(4):347-361.
38. Gatenby R A & Gillies R J (2004) Why do cancers have high aerobic glycolysis? Nat Rev Cancer 4(11):891-899.
39. Ebos J M, et al. (2009) Accelerated metastasis after short-term treatment with a potent inhibitor of tumor angiogenesis. Cancer Cell 15(3):232-239.
40. Paez-Ribes M, et al. (2009) Antiangiogenic therapy elicits malignant progression of tumors to increased local invasion and distant metastasis. Cancer Cell 15(3):220-231.
41. Caino M C, et al. (2013) Metabolic stress regulates cytoskeletal dynamics and metastasis of cancer cells. J Clin Invest 123(7):2907-2920.
42. Kang B H, et al. (2009) Combinatorial drug design targeting multiple cancer signaling networks controlled by mitochondrial Hsp90. J Clin Invest 119(3):454-464.
43. Ortensi B, et al. (2012) Rai is a new regulator of neural progenitor migration and glioblastoma invasion. Stem Cells 30(5):817-832
44. Vaira V, et al. (2012) miR-296 regulation of a cell polarity-cell plasticity module controls tumor progression. Oncogene 31(1):27-38.
45. Hinz B, Alt W, Johnen C, Herzog V, & Kaiser H-W (1999) Quantifying Lamella Dynamics of Cultured Cells by SACED, a New Computer-Assisted Motion Analysis. Exp Cell Res 251(1):234-243.

1. A method of reducing, inhibiting or preventing cancer metastasis comprising blocking movement of mitochondria within the cancer cell.

2. The method according to claim 1, further comprising interrupting or preventing oxidative phosphorylation pathways or respiration pathways in the cancer cell.

3. The method according to claim 1, wherein said mitochondrial movement is induced by contact of the cell with PI3K inhibitors or antagonists.

4. The method according to claim 1, further comprising down-regulating, inhibiting, suppressing or eliminating the expression or activity of MFN-2 in a cancer cell in the presence of a PI3K inhibitor or antagonist.

5. The method according to claim 1, further comprising down-regulating, inhibiting, suppressing or eliminating the expression or activity of TRAP-1 in the presence of a PI3K inhibitor or antagonist.

6. The method according to claim 1, further comprising down-regulating, inhibiting, suppressing or eliminating the expression or activity of mTOR in the presence of a PI3K inhibitor or antagonist.

7. The method according to claim 1, further comprising down-regulating, inhibiting, suppressing or eliminating the expression or activity of FAK in the presence of a PI3K inhibitor or antagonist.

8. The method according to claim 1, further comprising down-regulating, inhibiting, suppressing or eliminating the expression or activity of AMK in the presence of a PI3K inhibitor or antagonist.

9. The method according to claim 1, further comprising down-regulating, inhibiting, suppressing or eliminating the expression or activity of mito-Complex I in the presence of a PI3K inhibitor or antagonist.

10. The method according to claim 1, further comprising down-regulating, inhibiting, suppressing or eliminating the expression or activity of mito-Complex III in the presence of a PI3K inhibitor or antagonist.

11. The method according to claim 1, further comprising down-regulating, inhibiting, suppressing or eliminating the expression or activity of mito-Complex V in the presence of a PI3K inhibitor or antagonist.

12. The method according to claim 1, further comprising down-regulating, inhibiting, suppressing or eliminating the expression or activity of mitochondrial uncoupler CCCP in the presence of a PI3K inhibitor or antagonist.

13. The method according to claim 1, further comprising down-regulating, inhibiting, suppressing or eliminating the expression or activity of Aktl in the presence of a PI3K inhibitor or antagonist.

14. The method according to claim 1, further comprising down-regulating, inhibiting, suppressing or eliminating the expression or activity of Akt2 in the presence of a PI3K inhibitor or antagonist.

15. A method of improving therapeutic outcome in a cancer patient receiving PI3K inhibitors or antagonists comprising administering to said patient a composition or regimen that blocks oxidative phosphorylation or respiration of the cell.

16. The method according to claim 15, wherein said oxidative phosphorylation or respiration blocking therapy is administered concurrently with the PI3K therapy.

17. The method according to claim 15, wherein said oxidative phosphorylation or respiration blocking therapy is administered sequentially with the PI3K therapy.

18. The method according to claim 15, wherein said oxidative phosphorylation or respiration blocking therapy is administered at specific time points within the PI3K therapeutic regimen.

19. A method of screening molecules comprising:
a. contacting a mammalian cancer or tumor cell culture demonstrating mitochondrial trafficking with a test molecule;
b. culturing the cell and examining same for movement of mitochondria; and
c. measuring the oxidative phosphorylation of an AKT pathway, an mTOR pathway or a FAK pathway or respiration of the cell;
wherein a decrease or inhibition in mitochondrial trafficking, oxidative phosphorylation or respiration within the cell indicates that the test molecule has an anti-cancer or anti-tumor effect and
wherein the detection of, or increase in, mitochondrial trafficking, or increase in oxidative phosphorylation or respiration of certain pathways within the cell indicates that the test molecule has a carcinogenic, metastatic, or tumorigenic effect.

20. The method according to claim 19, wherein the mitochondrial trafficking is PI3K antagonist-induced.
